SEQUENCE LISTING (5 sequences)

SEQ ID NO: 1 - protein, 31 aa, Homo sapiens
HAEGTFTSDVSSYLEGQAAKEFIAWLVKGRG

SEQ ID NO: 2 - protein, 39 aa, Heloderma suspectum
HGEGTFTSDLSKQMEEEAVRLFIEWLKNGGPSSGAPPPS

SEQ ID NO: 3 - protein, 40 aa, Artificial Sequence
  Synthetic
XXEGTFTSDXSXXXEXXAXXXFIXWLXXXXXXXXXXXXXX

SEQ ID NO: 4 - protein, 32 aa, Artificial Sequence
  Synthetic
XXEGTFTSDVSXYLEXXAAXEFIXWLVXXXXX

SEQ ID NO: 5 - protein, 44 aa, Artificial Sequence
  Synthetic
HGEGTFTSDLSKQMEEEAVRLFIEWLKNGGPSSGAPPSKKKKKK

(19) United States
(12) Patent Application Publication
(10) Pub. No.: US 2010/0029903 A1
(43) Pub. Date: Feb. 4, 2010

(54) Polypeptide Protracting Tags

(75) Inventors: Kilian Waldemar Conde Frieboes, Maaloev (DK); Florencio Zaragoza Dorwald, Smoerum (DK); Janos Tibor Kodra, Copenhagen (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/097,194

(22) PCT Filed: Dec. 13, 2006
(86) PCT No.: PCT/EP2006/069656

§ 371 Date: Nov. 10, 2008

(30) Foreign Application Priority Data
Dec. 14, 2005 (EP) 05112134.1

(51) Int. Cl. C07K 14/605; C07K 14/00; C07F 9/38; C07F 9/09

(52) U.S. Cl. 530/308; 530/324; 562/24; 558/179; 558/172

(57) ABSTRACT

The invention provides novel compounds comprising a protracting tag linked to therapeutically active compounds.

### FIELD OF THE INVENTION

The present invention relates to novel compounds comprising a protracting tag linked to therapeutically active compounds, the novel protracting tags, methods for preparing the compounds and the medical applications of such compounds.

### BACKGROUND OF THE INVENTION

It is often desirable to maintain well-defined concentrations of a given compound in the blood stream for a long time. This would for instance be the case when an immunogen is administered and a strong immune response is desired, or when a therapeutic target has to be exposed continuously to a therapeutic agent for a long time. Currently there are no universally applicable strategies to enhance the plasma half-life of any type of compound.

The number of known endogenous polypeptides with interesting biological activities is growing rapidly, also as a result of the ongoing exploration of the human genome. Due to their biological activities, many of these polypeptides could in principle be used as therapeutic agents. Endogenous peptides are, however, not always suitable as drug candidates because these peptides often have half-lives of few minutes due to rapid degradation by peptidases and/or due to renal filtration and excretion in the urine. The half-life of polypeptides in human plasma varies strongly (from a few minutes to more than one week). Similarly, the half-life of small molecule drugs is also highly variable. The reason for this strong variability of plasma half-lives of peptides, proteins, or other compounds is, however, not well understood.

Serum albumin has a half-life of more than one week, and one approach to increasing the plasma half-life of peptides has been to derivatise the peptides with a chemical entity that binds to serum albumin.

Knudsen et al. (J. Med. Chem. 2000, 43, 1664-1669) have shown that acylated GLP-1 peptides exhibit high receptor potency and a tenfold increase of plasma half-life in pigs.

Zobel et al. (Bioorg. Med. Chem. Lett. 2003, 13, 1513-1515) have shown that the plasma half-life of an anticoagulant peptide in rabbits increased by 10-50 fold on derivatisation of the amino terminus with phosphate-based small molecules binding to serum albumin.

WO 2005/028516 relates to a new drug delivery system, based on novel compounds with high affinity to plasma proteins (=affinity tags). These affinity tags can be linked to therapeutically active compounds and thereby enhance their half-life in plasma by reversible binding to plasma proteins.

### SUMMARY OF THE INVENTION

According to the present invention there is provided a compound of the general formula I:

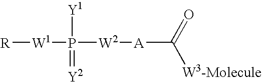

wherein
A represents —(CH₂)₁₋₂₀— in which one or more methylene groups are optionally replaced by a diradical selected from the group consisting of —O—, —S—, —NH— and —CH═CH— and which is optionally substituted with one or more substituent(s) selected from the group consisting of C₁₋₆-alkyl, C₁₋₆-alkoxy or carboxy,
R represents C₁₋₂₀-alkyl- in which one or more methylene groups are optionally replaced by a diradical selected from the group consisting of —O—, —S—, —NH— and —CH═CH— and which is optionally substituted with one or more substituent(s) selected from the group consisting of aryl, haloaryl, cyanoaryl, heteroaryl, C₃₋₁₀-cycloalkyl, aryl-C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl; or C₃₋₁₀-cycloalkyl- optionally substituted with one or more substituent(s) selected from the group consisting of aryl, haloaryl, cyanoaryl, heteroaryl, C₃₋₁₀-cycloalkyl, carboxyl, 5-tetrazolyl, acylaminosulfonyl, sulfonylaminocarbonyl, and a straight or branched C₁₋₆-alkyl,
W¹ and W² independently represent —O—, —CH₂— or —S—,
Y¹ represents —OH or —SH,
Y² represents ═O or ═S,
W³ represents a bond or a spacer, and
the term “molecule” represents a fragment obtained by formal abstraction of a hydrogen atom from an amino group, a hydroxy group, or a mercapto group of a therapeutically effective polypeptide,
with the proviso that at least either A represents —(CH₂)₁₁₋₂₀— in which one or more methylene groups are optionally replaced by a diradical selected from the group consisting of —O—, —S—, —NH— and —CH═CH— and which is optionally substituted with one or more substituent(s) selected from the group consisting of C₁₋₆-alkyl, C₁₋₆-alkoxy or carboxy or R represents C₁₁₋₂₀-alkyl- in which one or more methylene groups are optionally replaced by a diradical selected from the group consisting of —O—, —S—, —NH— and —CH═CH— and which is optionally substituted with one or more substituent(s) selected from the group consisting of aryl, haloaryl, cyanoaryl, heteroaryl, C₃₋₁₀-cycloalkyl, aryl-C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl.

According to the present invention there is also provided a compound of the general formula II

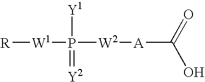

wherein
A represents —(CH₂)₁₋₂₀— in which one or more methylene groups are optionally replaced by a diradical selected from the group consisting of —O—, —S—, —NH— and —CH═CH— and which is optionally substituted with one or more substituent(s) selected from the group consisting of C₁₋₆-alkyl, C₁₋₆-alkoxy or carboxy,
R represents C₁₋₂₀-alkyl- in which one or more methylene groups are optionally replaced by a diradical selected from the group consisting of —O—, —S—, —NH— and —CH═CH— and which is optionally substituted with one or more substituent(s) selected from the group consisting of aryl, haloaryl, cyanoaryl, heteroaryl, C₃₋₁₀-cycloalkyl, aryl-C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl; or C₃₋₁₀-cycloalkyl- optionally substituted with one or more substituent(s) selected from the group consisting of aryl, haloaryl, cyanoaryl, heteroaryl, C₃₋₁₀-cycloalkyl, carboxyl, 5-tetrazolyl, acylaminosulfonyl, sulfonylaminocarbonyl, and a straight or branched C₁₋₆-alkyl,
W¹ and W² independently represent —O—, —CH₂— or —S—,
Y¹ represents —OH or —SH, and
Y² represents ═O or ═S,
with the proviso that at least either A represents —(CH₂)₁₁₋₂₀— in which one or more methylene groups are optionally replaced by a diradical selected from the group consisting of —O—, —S—, —NH— and —CH═CH— and which is optionally substituted with one or more substituent(s) selected from the group consisting of C₁₋₆-alkyl, C₁₋₆-alkoxy or carboxy, or R represents C₁₁₋₂₀-alkyl- in which one or more methylene groups are optionally replaced by a diradical selected from the group consisting of —O—, —S—, —NH— and —CH═CH— and which is optionally substituted with one or more substituent(s) selected from the group consisting of aryl, haloaryl, cyanoaryl, heteroaryl, C₃₋₁₀-cycloalkyl, aryl C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl.

The present invention also relates to pharmaceutical compositions comprising a compound according to the present invention and the use of compounds according to the present invention for preparing medicaments for treating diseases.

### DEFINITIONS

In the present specification, the following terms have the indicated meaning:

The term “molecule” as used herein represents a fragment obtained by formal abstraction of a hydrogen atom from an amino group, a hydroxy group, or a mercapto group of a therapeutically effective polypeptide.

The term “therapeutically effective polypeptide” or “therapeutic polypeptides” as used herein refers to a polypeptide able to cure, alleviate or partially arrest the clinical manifestations of a given disease and its complications.

In a further aspect of the invention the term “therapeutically effective polypeptide” or “therapeutic polypeptides” as used herein means a polypeptide which is being developed for therapeutic use, or which has been developed for therapeutic use.

An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

The term “polypeptide” or “peptide” as used herein means a compound composed of at least five constituent amino acids connected by peptide bonds. The constituent amino acids may be from the group of the amino acids encoded by the genetic code and they may natural amino acids which are not encoded by the genetic code, as well as synthetic amino acids. Natural amino acids which are not encoded by the genetic code are e.g. hydroxyproline, γ-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (α-aminoisobutyric acid), Abu (α-aminobutyric acid), Tle (tert-butylglycine), β-alanine, 3-aminomethyl benzoic acid, anthranilic acid.

The term “plasma half-life” as used herein refers to the time required for the concentration of a given compound present in the plasma of a living mammal, such as a human, to decrease to one half of its original concentration.

The term “being bound to albumin” as used herein refers to the ability of the compound according to the invention of binding in plasma to the plasma protein serum albumin.

The term “analog” as used herein refers to a polypeptide in which less than 30% of the amino acids of the original polypeptide have been removed or replaced by other amino acids (including stereoisomeric, unnatural or chemically modified amino acids) or have been chemically modified, for instance by acylation or alkylation of the side chain. The term “analog” also refers to polypeptides in which the N-terminal amino group has been removed, alkylated with C₁₋₆-alkyl, or acylated with lower alkanoic, arylalkanoic, heteroarylalkanoic, or benzoic acids. The term “analog” also includes polypeptides in which the C-terminal carboxyl group has been removed or converted to an amide by condensation with ammonia, C₁₋₆-alkyl amines, di-C₁₋₆-alkyl amines, aziridine, azetidine, pyrrolidine, piperidine, or azepine. The term “analog” also includes polypeptides in which the disulfide functionalities between two or more cysteine groups have been reduced or the connectivity between two or more cystein groups has been modified.

The term “derivative” as used herein in relation to a polypeptide refers to a chemically modified peptide or an analogue thereof, wherein at least one substituent is not present in the unmodified peptide or an analogue thereof, i.e. a peptide which has been covalently modified. Typical modifications are amides, carbohydrates, C₁₋₆-alkyl groups, acyl groups, esters and the like. An example of a derivative of GLP-1(7-37) is Arg³⁴Lys²⁶(N^{ε}-(γ-Glu(N^{α}-hexadecanoyl)))-GLP-1(7-37).

The term “unnatural amino acid” as used herein refers to any compound comprising at least one primary or secondary amino group and at least one carboxyl group, without being L-alanine, L-arginine, L-asparagine, L-aspartic acid, L-cysteine, L-glutamine, L-glutamic acid, L-glycine, L-histidine, L-isoleucine, L-leucine, L-lysine, L-methionine, L-phenylalanine, proline, L-serine, L-threonine, L-tryptophan, L-tyrosine, or L-valine.

As used herein, the term “solvate” is a complex of defined stoichiometry formed by a solute (in casu, a compound according to the present invention) and a solvent. Solvents may be, by way of example, water, ethanol, or acetic acid.

In addition the following abbreviations have the meanings given

| | |
|---|---|
| Ac | Acetyl |
| 4-Abu | 4-aminobutyric acid |
| Ala | Alanine |
| Arg | Arginine |
| Arg(Pmc) | 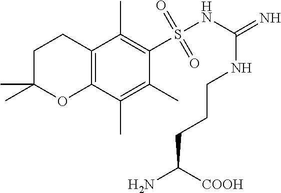 |
| Asn | Asparagine |
| Asn(alkyl) | 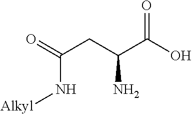 |
| Asn(aryl) | 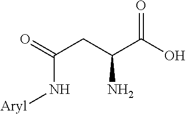 |
| Asp | aspartic acid |
| Boc | tert-butyloxycarbonyl |
| Cys | Cysteine |
| D-Phe | D form of phenylalanine |
| Fmoc | 9-fluorenylmethoxycarbonyl |
| F-Pro | 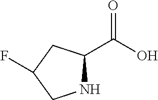 |
| Gly | Glycine |
| Gln | Glutamine |
| Gln(alkyl) | 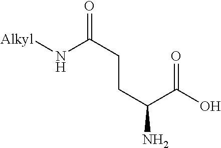 |
| Gln(aryl) | 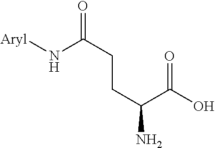 |
| Glu | glutamic acid |
| His | Histidine |
| homoArg | 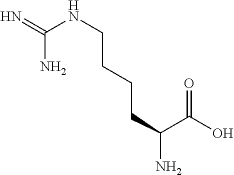 |
| homoCys | 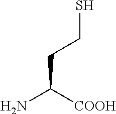 |
| Hyp | 4-hydroxyproline |
| Ile | Isoleucine |
| Leu | Leucine |
| Lys | Lysine |
| Met | Methionine |
| Met(O) | 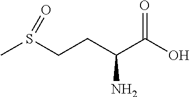 |
| Met(O₂) | 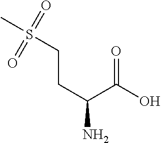 |
| mPEG2000 | methoxypolyethylenglycol (average molecular weight of |
| | 2000 Dalton) |
| Mtt | 4-methyltrityl |
| 2Nal | 2-Naphthyl alanine |
| Nle | Norleucine |
| Orn | Ornithine |
| Phe | Phenylalanine |
| Pro | Proline |
| 2-PyAla | 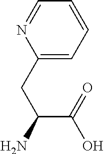 |
| 3-PyAla | 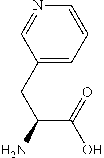 |
| 4-PyAla | 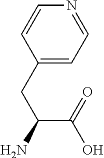 |
| Ser | Serine |
| Thr | Threonine |
| Tic | (S)-1,2,3,4-tetrahydroisoquinoline-3-carboxylic acid |
| tBu | tert-butyl |
| Thr | Threonine |
| Trp | Tryptophane |
| Val | Valine |

When two amino acids are said to be bridged it is intended to indicate that functional groups in the side chains of the two amino acids have reacted to form a covalent bond. In the present context, the term “agonist” is intended to indicate a substance that activates the receptors.

In the present context, the term “antagonist” is intended to indicate a substance that neutralizes or counteracts the effect of an agonist.

In the present context, the term “pharmaceutically acceptable salt” is intended to indicate salts which are not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

In the present context, AA(X), wherein AA indicates an amino acid, is intended to indicate that X is attached to the functional group in the side chain of the amino acid.

The term “GLP-1(7-37)” refers to a peptide with the amino acid sequence

| | |
|---|---|
| HAEGTFTSDVSSYLEGQAAKEFIAWLVKGRG. | (SEQ ID No. 1) |

The term “GLP-1 peptide” as used herein refers to GLP-1(7-37), a GLP-1 analog, a GLP-1 derivative or a derivative of a GLP-1 analog. In one aspect the GLP-1 peptide is an insulinotropic agent.

The term “exendin-4(1-39)” refers to a peptide with the amino acid sequence

(SEQ ID No. 2)

HGEGTFTSDLSKQMEEEAVRLFIEWLKNGGPSSGAPPPS.

The term “insulinotropic agent” as used herein refers to a compound which is an agonist of the human GLP-1 receptor, i.e. a compound which stimulates the formation of cAMP in a suitable medium containing the human GLP-1 receptor. The potency of an insulinotropic agent is determined by calculating the EC50 value from the dose-response curve as described below.

A stable transfected cell line has been prepared at Novo Nordisk and a high expressing clone was selected for screening. The cells were grown at 5% CO₂ in DMEM, 50% FCS, 1% Pen/Strep and 0.5 mg/ml G418.

Cells at approximate 80% confluence were washed 2× with PBS and harvested with Versene, centrifuged 5 min at 1000 rpm and the supernatant removed. The additional steps were all made on ice. The cell pellet was homogenized by the Ultrathurax for 20-30 s. in 10 ml of Buffer 1 (20 mM Na-HEPES, 10 mM EDTA, pH=7.4), centrifuged 15 min at 20.000 rpm and the pellet resuspended in 10 ml of Buffer 2 (20 mM Na-HEPES, 0.1 mM EDTA, pH=7.4). The suspension was homogenized for 20-30 s and centrifuged 15 min at 20.000 rpm. Suspension in Buffer 2, homogenization and centrifugation was repeated once and the membranes were resuspended in Buffer 2 and ready for further analysis or stored at −80° C.

The functional receptor assay was carried out by measuring the peptide-induced cAMP production by The AlphaScreen™ cAMP Technology from Perkin Elmer Life Sciences. The basic principle of The AlphaScreen Technology is a competition between endogenous cAMP and exogenously added biotin-cAMP. The capture of cAMP is achieved by using a specific antibody conjugated to acceptor beads. Formed cAMP was counted and measured at a AlphaFusion Microplate Analyzer. The EC₅₀ values were calculated using the Graph-Pad Prisme software.

The binding assay was performed with purified plasma membranes containing the human GLP-1 receptor. The plasma membranes containing the receptors were purified from stably expressing BHK tk-ts 13 cells. The membranes were diluted in Assay Buffer (50 mM HEPES, 5 mM EGTA, 5 mM MgCl₂, 0.0050% Tween 20, pH=7.4) to a final concentration of 0.2 mg/ml of protein and distributed to 96-well microtiter plates precoated with 0.3% PEI. Membranes in the presence of 0.05 nM [¹²⁵I]GLP-1, unlabelled ligands in increasing concentrations and different HSA concentrations (0.005%, 0.05%, and 2%) were incubated 2 h at 30° C. After incubation, unbound ligands were separated from bound ligands by filtration through a vacuum-manifold followed by 2×100 μl washing with ice cold assay buffer. The filters were dried overnight at RT, punched out and quantified in a γ-counter.

The term “DPP-IV protected” as used herein referring to a polypeptide means a polypeptide which has been chemically modified in order to render said compound resistant to the plasma peptidase dipeptidyl aminopeptidase-4 (DPP-IV). The DPP-IV enzyme in plasma is known to be involved in the degradation of several peptide hormones, e.g. GLP-1, GLP-2, Exendin-4 etc. Thus, a considerable effort is being made to develop analogues and derivatives of the polypeptides susceptible to DPP-IV mediated hydrolysis in order to reduce the rate of degradation by DPP-IV.

Resistance of a peptide to degradation by dipeptidyl aminopeptidase IV is determined by the following degradation assay:

Aliquots of the peptide (5 nmol) are incubated at 37° C. with 1 μL of purified dipeptidyl aminopeptidase IV corresponding to an enzymatic activity of 5 mU for 10-180 minutes in 100 μL of 0.1 M triethylamine-HCl buffer, pH 7.4. Enzymatic reactions are terminated by the addition of 5 μL of 10% trifluoroacetic acid, and the peptide degradation products are separated and quantified using HPLC analysis. One method for performing this analysis is: The mixtures are applied onto a Vydac C18 widepore (30 nm pores, 5 μm particles) 250×4.6 mm column and eluted at a flow rate of 1 ml/min with linear stepwise gradients of acetonitrile in 0.1% trifluoroacetic acid (0% acetonitrile for 3 min, 0-24% acetonitrile for 17 min, 24-48% acetonitrile for 1 min) according to Siegel et al., Regul. Pept. 1999; 79:93-102 and Mentlein et al. Eur. J. Biochem. 1993; 214:829-35. Peptides and their degradation products may be monitored by their absorbance at 220 nm (peptide bonds) or 280 nm (aromatic amino acids), and are quantified by integration of their peak areas related to those of standards. The rate of hydrolysis of a peptide by dipeptidyl aminopeptidase IV is estimated at incubation times which result in less than 10% of the peptide being hydrolysed.

The term “diradical” refers to a molecular moiety with two unshared electrons. A diradical according to this definition may be used to covalently link two radicals together.

The term “halogen” or “halo” means fluorine, chlorine, bromine or iodine.

The term “hydroxyl” shall mean the radical —OH.

The term “cyano” shall mean the radical —CN.

The term “mercapto” shall mean the radical —SH.

The term “sulfonyl” shall mean the radical —S(═O)₂—.

The term “carboxyl” shall mean the radical —(C═O)OH.

The term “amino” shall mean the radical —NH₂.

The use of prefixes of the structure: C_{x-y}-alkyl, C_{x-y}-alkenyl, C_{x-y}-alkynyl or C_{x-y}-cycloalkyl designates a radical of the designated type having from x to y carbon atoms.

The term “C₁₋₆-alkyl” as used herein represents a saturated, branched or straight hydrocarbon group having from 1 to 6 carbon atoms, e.g. C₁₋₃-alkyl, C₁₋₄-alkyl, C₁₋₆-alkyl, C₂₋₆-alkyl, C₃₋₆-alkyl. Representative examples are methyl, ethyl, propyl (e.g. prop-1-yl, prop-2-yl (or iso-propyl)), butyl (e.g. 2-methylprop-2-yl (or tert-butyl), but-1-yl, but-2-yl), pentyl (e.g. pent-1-yl, pent-2-yl, pent-3-yl), 2-methylbut-1-yl, 3-methylbut-1-yl, hexyl (e.g. hex-1-yl), and the like.

The term “C₁₋₂₀-alkyl”, “C₁₁₋₂₀-alkyl” and “C₁₁₋₁₈-alkyl”, as used herein represents a saturated, branched or straight hydrocarbon group having from respectively 1 to 20 carbon atoms, 11 to 20 carbon atoms and 11 to 18 carbon atoms e.g. C₁₋₃-alkyl, C₁₋₄-alkyl, C₁₋₆-alkyl, C₂₋₆-alkyl, C₃₋₆-alkyl, C₁₋₈-alkyl, C₁₋₁₀-alkyl, C₃₋₁₂-alkyl, C₆₋₁₂-alkyl, C₁₁₋₂₀-alkyl, C₁₁₋₁₈-alkyl and the like. Representative examples are methyl, ethyl, propyl (e.g. prop-1-yl, prop-2-yl (or iso-propyl)), butyl (e.g. 2-methylprop-2-yl (or tert-butyl), but-1-yl, but-2-yl), pentyl (e.g. pent-1-yl, pent-2-yl, pent-3-yl), 2-methylbut-1-yl, 3-methylbut-1-yl, hexyl (e.g. hex-1-yl), heptyl (e.g. hept-1-yl), octyl (e.g. oct-1-yl), nonyl (e.g. non-1-yl), and the like.

The term “alkenyl” as used herein without prefixes, refers to a straight or branched chain monovalent non-aromatic hydrocarbon radical having for instance from two to ten carbon atoms and at least one carbon-carbon double bond, for example C₂₋₈-alkenyl. Typical C₂₋₈-alkenyl groups include vinyl, 1-propenyl, 2-propenyl, iso-propenyl, 1,3-butadienyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 2,4-hexadienyl, and 5-hexenyl.

In the present context, the term alkynyl used without prefixes is intended to indicate a straight or branched chain non-aromatic monovalent hydrocarbon having at least one carbon-carbon triple bond and optionally one or more carbon-carbon double bonds having for instance from 2 to 10 carbon atoms. Examples of alkynyl include 2-propynyl, 2-butynyl and 1,3-hexadiene-5-ynyl.

In the present context, the term “alkanoyl” is intended to indicate a radical of the formula —C(O)—R′, wherein R′ is alkyl as indicated above such as e.g. C₁₋₄-alkanoyl.

The term “alkoxy” is intended to indicate a radical of the formula —O—R′, wherein R′ is alkyl as indicated above. In one aspect of the invention, “alkoxy” is C₁₋₆-alkoxy.

The term “C₁₋₆-alkoxy” as used herein refers to the radical C₁₋₆-alkyl-O—. Representative examples are methoxy, ethoxy, propoxy (e.g. 1-propoxy, 2-propoxy), butoxy (e.g. 1-butoxy, 2-butoxy, 2-methyl-2-propoxy), pentoxy (1-pentoxy, 2-pentoxy), hexoxy (1-hexoxy, 3-hexoxy), and the like.

The term “C₃₋₁₀-cycloalkyl” as used herein represents a saturated monocyclic carbocyclic ring having from 3 to 10 carbon atoms, e.g. C₃₋₆-alkyl, C₃₋₈-alkyl, C₃₋₁₀-alkyl, C₃₋₁₂-alkyl, C₃₋₁₅-alkyl, C₅₋₁₂-alkyl, C₆₋₁₂-alkyl, C₅₋₁₅-alkyl, C₆₋₁₅-alkyl, and the like. Representative examples are cyclopropyl, cyclobutyl, cyclopentyl, cyclohexyl, cycloheptyl, cyclooctyl, and the like. C₃₋₁₀-cycloalkyl is also intended to represent a saturated bicyclic carbocyclic ring having from 4 to 10 carbon atoms. Representative examples are decahydronaphthalenyl, bicyclo[3.3.0]octanyl, and the like. C₃₋₁₀-cycloalkyl is also intended to represent a saturated carbocyclic ring having from 3 to 10 carbon atoms and containing one or two carbon bridges. Representative examples are adamantyl, norbornanyl, nortricyclyl, bicyclo[3.2.1]octanyl, bicyclo[2.2.2]octanyl, tricyclo[5.2.1.0/2,6]decanyl, bicyclo[2.2.1]heptyl, and the like. C₃₋₁₀-cycloalkyl is also intended to represent a saturated carbocyclic ring having from 3 to 10 carbon atoms and containing one or more spiro atoms. Representative examples are spiro[2.5]octanyl, spiro[4.5]decanyl, and the like.

The term “aryl” as used herein is intended to include monocyclic, bicyclic or polycyclic carbocyclic aromatic rings. Representative examples are phenyl, naphthyl (e.g. naphth-1-yl, naphth-2-yl), anthryl (e.g. anthr-1-yl, anthr-9-yl), phenanthryl (e.g. phenanthr-1-yl, phenanthr-9-yl), and the like. Aryl is also intended to include monocyclic, bicyclic or polycyclic carbocyclic aromatic rings substituted with carbocyclic aromatic rings. Representative examples are biphenyl (e.g. biphenyl-2-yl, biphenyl-3-yl, biphenyl-4-yl), phenylnaphthyl (e.g. 1-phenylnaphth-2-yl, 2-phenylnaphth-1-yl), and the like. Aryl is also intended to include partially saturated bicyclic or polycyclic carbocyclic rings with at least one unsaturated moiety (e.g. a benzo moiety). Representative examples are indanyl (e.g. indan-1-yl, indan-5-yl), indenyl (e.g. inden-1-yl, inden-5-yl), 1,2,3,4-tetrahydronaphthyl (e.g. 1,2,3,4-tetrahydronaphth-1-yl, 1,2,3,4-tetrahydronaphth-2-yl, 1,2,3,4-tetrahydronaphth-6-yl), 1,2-dihydronaphthyl (e.g. 1,2-dihydronaphth-1-yl, 1,2-dihydronaphth-4-yl, 1,2-dihydronaphth-6-yl), fluorenyl (e.g. fluoren-1-yl, fluoren-4-yl, fluoren-9-yl), and the like. Aryl is also intended to include partially saturated bicyclic or polycyclic carbocyclic aromatic rings containing one or two bridges. Representative examples are benzonorbornyl (e.g. benzonorborn-3-yl, benzonorborn-6-yl), 1,4-ethano-1,2,3,4-tetrahydronapthyl (e.g. 1,4-ethano-1,2,3,4-tetrahydronapth-2-yl, 1,4-ethano-1,2,3,4-tetrahydronapth-10-yl), and the like. Aryl is also intended to include partially saturated bicyclic or polycyclic carbocyclic aromatic rings containing one or more spiro atoms. Representative examples are spiro[cyclopentane-1,1′-indane]-4-yl, spiro[cyclopentane-1,1′-indene]-4-yl, spiro[piperidine-4,1′-indane]-1-yl, spiro[piperidine-3,2′-indane]-1-yl, spiro[piperidine-4,2′-indane]-1-yl, spiro[piperidine-4,1′-indane]-3′-yl, spiro[pyrrolidine-3,2′-indane]-1-yl, spiro[pyrrolidine-3,1′-(3′,4′-dihydronaphthalene)]-1-yl, spiro[piperidine-3,1′-(3′,4′-dihydronaphthalene)]-1-yl, spiro[piperidine-4,1′-(3′,4′-dihydronaphthalene)]-1-yl, spiro[imidazolidine-4,2′-indane]-1-yl, spiro[piperidine-4,1′-indene]-1-yl, and the like.

The term “bridge” as used herein represents a connection in a saturated or partly saturated ring between two atoms of such ring that are not neighbours through a chain of 1 to 3 atoms selected from carbon, nitrogen, oxygen and sulfur. Representative examples of such connecting chains are —CH₂—, —CH₂CH₂—, —CH₂NHCH₂—, —CH₂CH₂CH₂—, —CH₂OCH₂—, and the like.

The term “spiro atom” as used herein represents a carbon atom in a saturated or partly saturated ring that connects both ends of a chain of 3 to 7 atoms selected from carbon, nitrogen, oxygen and sulfur. Representative examples are —(CH₂)₅—, —(CH₂)₃—, —(CH₂)₄—, —CH₂NHCH₂CH₂—, —CH₂CH₂NHCH₂CH₂—, —CH₂NHCH₂CH₂CH₂—, —CH₂CH₂OCH₂—, —OCH₂CH₂O—, and the like.

The term “heteroaryl” as used herein is intended to include monocyclic heterocyclic aromatic rings containing one or more heteroatoms selected from nitrogen, oxygen, sulfur, SO and S(═O)₂. Representative examples are pyrrolyl (e.g. pyrrol-1-yl, pyrrol-2-yl, pyrrol-3-yl), furanyl (e.g. furan-2-yl, furan-3-yl), thienyl (e.g. thien-2-yl, thien-3-yl), oxazolyl (e.g. oxazol-2-yl, oxazol-4-yl, oxazol-5-yl), thiazolyl (e.g. thiazol-2-yl, thiazol-4-yl, thiazol-5-yl), imidazolyl (e.g. imidazol-2-yl, imidazol-4-yl, imidazol-5-yl), pyrazolyl (e.g. pyrazol-1-yl, pyrazol-3-yl, pyrazol-5-yl), isoxazolyl (e.g. isoxazol-3-yl, isoxazol-4-yl, isoxazol-5-yl), isothiazolyl (e.g. isothiazol-3-yl, isothiazol-4-yl, isothiazol-5-yl), 1,2,3-triazolyl (e.g. 1,2,3-triazol-1-yl, 1,2,3-triazol-4-yl, 1,2,3-triazol-5-yl), 1,2,4-triazolyl (e.g. 1,2,4-triazol-1-yl, 1,2,4-triazol-3-yl, 1,2,4-triazol-5-yl), 1,2,3-oxadiazolyl (e.g. 1,2,3-oxadiazol-4-yl, 1,2,3-oxadiazol-5-yl), 1,2,4-oxadiazolyl (e.g. 1,2,4-oxadiazol-3-yl, 1,2,4-oxadiazol-5-yl), 1,2,5-oxadiazolyl (e.g. 1,2,5-oxadiazol-3-yl, 1,2,5-oxadiazol-4-yl), 1,3,4-oxadiazolyl (e.g. 1,3,4-oxadiazol-2-yl, 1,3,4-oxadiazol-5-yl), 1,2,3-thiadiazolyl (e.g. 1,2,3-thiadiazol-4-yl, 1,2,3-thiadiazol-5-yl), 1,2,4-thiadiazolyl (e.g. 1,2,4-thiadiazol-3-yl, 1,2,4-thiadiazol-5-yl), 1,2,5-thiadiazolyl (e.g. 1,2,5-thiadiazol-3-yl, 1,2,5-thiadiazol-4-yl), 1,3,4-thiadiazolyl (e.g. 1,3,4-thiadiazol-2-yl, 1,3,4-thiadiazol-5-yl), tetrazolyl (e.g. tetrazol-1-yl, tetrazol-5-yl), pyranyl (e.g. pyran-2-yl), pyridinyl (e.g. pyridine-2-yl, pyridine-3-yl, pyridine-4-yl), pyridazinyl (e.g. pyridazin-2-yl, pyridazin-3-yl), pyrimidinyl (e.g. pyrimidin-2-yl, pyrimidin-4-yl, pyrimidin-5-yl), pyrazinyl, 1,2,3-triazinyl, 1,2,4-triazinyl, 1,3,5-triazinyl, thiadiazinyl, azepinyl, azecinyl, and the like. Heteroaryl is also intended to include bicyclic heterocyclic aromatic rings containing one or more heteroatoms selected from nitrogen, oxygen, sulfur, S(═O) and S(═O)₂. Representative examples are indolyl (e.g. indol-1-yl, indol-2-yl, indol-3-yl, indol-5-yl), isoindolyl, benzofuranyl (e.g. benzo[b]furan-2-yl, benzo[b]furan-3-yl, benzo[b]furan-5-yl, benzo[c]furan-2-yl, benzo[c]furan-3-yl, benzo[c]furan-5-yl), benzothienyl (e.g. benzo[b]thien-2-yl, benzo[b]thien-3-yl, benzo[b]thien-5-yl, benzo[c]thien-2-yl, benzo[c]thien-3-yl, benzo[c]thien-5-yl), indazolyl (e.g. indazol-1-yl, indazol-3-yl, indazol-5-yl), indolizinyl (e.g. indolizin-1-yl, indolizin-3-yl), benzopyranyl (e.g. benzo[b]pyran-3-yl, benzo[b]pyran-6-yl, benzo[c]pyran-1-yl, benzo[c]pyran-7-yl), benzimidazolyl (e.g. benzimidazol-1-yl, benzimidazol-2-yl, benzimidazol-5-yl), benzothiazolyl (e.g. benzothiazol-2-yl, benzothiazol-5-yl), benzisothiazolyl, benzoxazolyl, benzisoxazolyl, benzoxazinyl, benzotriazolyl, naphthyridinyl (e.g. 1,8-naphthyridin-2-yl, 1,7-naphthyridin-2-yl, 1,6-naphthyridin-2-yl), phthalazinyl (e.g. phthalazin-1-yl, phthalazin-5-yl), pteridinyl, purinyl (e.g. purin-2-yl, purin-6-yl, purin-7-yl, purin-8-yl, purin-9-yl), quinazolinyl (e.g. quinazolin-2-yl, quinazolin-4-yl, quinazolin-6-yl), cinnolinyl, quinoliny (e.g. quinolin-2-yl, quinolin-3-yl, quinolin-4-yl, quinolin-6-yl), isoquinolinyl (e.g. isoquinolin-1-yl, isoquinolin-3-yl, isoquinolin-4-yl), quinoxalinyl (e.g. quinoxalin-2-yl, quinoxalin-5-yl), pyrrolopyridinyl (e.g. pyrrolo[2,3-b]pyridinyl, pyrrolo[2,3-c]pyridinyl, pyrrolo[3,2-c]pyridinyl), furopyridinyl (e.g. furo[2,3-b]pyridinyl, furo[2,3-c]pyridinyl, furo[3,2-c]pyridinyl), thienopyridinyl (e.g. thieno[2,3-b]pyridinyl, thieno[2,3-c]pyridinyl, thieno[3,2-c]pyridinyl), imidazopyridinyl (e.g. imidazo[4,5-b]pyridinyl, imidazo[4,5-c]pyridinyl, imidazo[1,5-a]pyridinyl, imidazo[1,2-a]pyridinyl), imidazopyrimidinyl (e.g. imidazo[1,2-a]pyrimidinyl, imidazo[3,4-a]pyrimidinyl), pyrazolopyridinyl (e.g. pyrazolo[3,4-b]pyridinyl, pyrazolo[3,4-c]pyridinyl, pyrazolo[1,5-a]pyridinyl), pyrazolopyrimidinyl (e.g. pyrazolo[1,5-a]pyrimidinyl, pyrazolo[3,4-d]pyrimidinyl), thiazolopyridinyl (e.g. thiazolo[3,2-d]pyridinyl), thiazolopyrimidinyl (e.g. thiazolo[5,4-d]pyrimidinyl), imdazothiazolyl (e.g. imidazo[2,1-b]thiazolyl), triazolopyridinyl (e.g. triazolo[4,5-b]pyridinyl), triazolopyrimidinyl (e.g. 8-azapurinyl), and the like. Heteroaryl is also intended to include polycyclic heterocyclic aromatic rings containing one or more heteroatoms selected from nitrogen, oxygen, sulfur, S(═O) and S(═O)₂. Representative examples are carbazolyl (e.g. carbazol-2-yl, carbazol-3-yl, carbazol-9-yl), phenoxazinyl (e.g. phenoxazin-10-yl), phenazinyl (e.g. phenazin-5-yl), acridinyl (e.g. acridin-9-yl, acridin-10-yl), phenothiazinyl (e.g. phenothiazin-10-yl), carbolinyl (e.g. pyrido[3,4-b]indol-1-yl, pyrido[3,4-b]indol-3-yl), phenanthrolinyl (e.g. phenanthrolin-5-yl), and the like. Heteroaryl is also intended to include partially saturated monocyclic, bicyclic or polycyclic heterocyclic rings containing one or more heteroatoms selected from nitrogen, oxygen, sulfur, S(═O) and S(═O)₂. Representative examples are pyrrolinyl, pyrazolinyl, imidazolinyl (e.g. 4,5-dihydroimidazol-2-yl, 4,5-dihydroimidazol-1-yl), indolinyl (e.g. 2,3-dihydroindol-1-yl, 2,3-dihydroindol-5-yl), dihydrobenzofuranyl (e.g. 2,3-dihydrobenzo[b]furan-2-yl, 2,3-dihydrobenzo[b]furan-4-yl), dihydrobenzothienyl (e.g. 2,3-dihydrobenzo[b]thien-2-yl, 2,3-dihydrobenzo[b]thien-5-yl), 4,5,6,7-tetrahydrobenzo[b]furan-5-yl), dihydrobenzopyranyl (e.g. 3,4-dihydrobenzo[b]pyran-3-yl, 3,4-dihydrobenzo[b]pyran-6-yl, 3,4-dihydrobenzo[c]pyran-1-yl, dihydrobenzo[c]pyran-7-yl), oxazolinyl (e.g. 4,5-dihydrooxazol-2-yl, 4,5-dihydrooxazol-4-yl, 4,5-dihydrooxazol-5-yl), isoxazolinyl, oxazepinyl, tetrahydroindazolyl (e.g. 4,5,6,7-tetrahydroindazol-1-yl, 4,5,6,7-tetrahydroindazol-3-yl, 4,5,6,7-tetrahydroindazol-4-yl, 4,5,6,7-tetrahydroindazol-6-yl), tetrahydrobenzimidazolyl (e.g. 4,5,6,7-tetrahydrobenzimidazol-1-yl, 4,5,6,7-tetrahydrobenzimidazol-5-yl), tetrahydroimidazo[4,5-c]pyridyl (e.g. 4,5,6,7-tetrahydroimidazo[4,5-c]pyrid-1-yl, 4,5,6,7-tetrahydroimidazo[4,5-c]pyrid-5-yl, 4,5,6,7-tetrahydroimidazo[4,5-c]pyrid-6-yl), tetrahydroquinolinyl (e.g. 1,2,3,4-tetrahydroquinolinyl, 5,6,7,8-tetrahydroquinolinyl), tetrahydroisoquinolinyl (e.g. 1,2,3,4-tetrahydroisoquinolinyl, 5,6,7,8-tetrahydroisoquinolinyl), tetrahydroquinoxalinyl (e.g. 1,2,3,4-tetrahydroquinoxalinyl, 5,6,7,8-tetrahydroquinoxalinyl), and the like. Heteroaryl is also intended to include partially saturated bicyclic or polycyclic heterocyclic rings containing one or more spiro atoms. Representative examples are spiro[isoquinoline-3,1′-cyclohexan]-1-yl, spiro[piperidine-4,1′-benzo[c]thiophen]-1-yl, spiro[piperidine-4,1′-benzo[c]furan]-1-yl, spiro[piperidine-4,3′-benzo[b]furan]-1-yl, spiro[piperidine-4,3′-coumarin]-1-yl, and the like.

The term “optionally substituted” as used herein means that the groups in question are either unsubstituted or substituted with one or more of the substituents specified. When the group(s) in question are substituted with more than one substituent the substituents may be the same or different.

Certain of the defined terms may occur more than once in the structural formulae, and upon such occurrence each term shall be defined independently of the other.

Certain of the defined terms may occur in combinations, and it is to be understood that the first mentioned radical is a substituent on the subsequently mentioned radical, where the point of substitution, i.e. the point of attachment to another part of the molecule, is on the last mentioned of the radicals.

“Haloaryl” as used herein refers to aryl, substituted one or more times at any carbon atom(s) with any halogen.

“Cyanoaryl” as used herein refers to aryl, substituted one or more times at any carbon atom(s) with a cyano-group.

“Aryl-C₃₋₁₀-cycloalkyl” as used herein refers to C₃₋₁₀-cycloalkyl, substituted one or more times at any carbon atom(s) with aryl.

“Diaryl-C₃₋₁₀-cycloalkyl” as used herein refers to C₃₋₁₀-cycloalkyl, substituted two times at any carbon atom(s) with aryl.

### DETAILED DESCRIPTION OF THE INVENTION

In one aspect of the invention there is provided a compound of the general formula I:

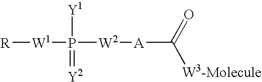

wherein
A represents —(CH₂)₁₋₂₀— in which one or more methylene groups are optionally replaced by a diradical selected from the group consisting of —O—, —S—, —NH— and —CH═CH— and which is optionally substituted with one or more substituent(s) selected from the group consisting of C₁₋₆-alkyl, C₁₋₆-alkoxy or carboxy,
R represents C₁₋₂₀-alkyl- in which one or more methylene groups are optionally replaced by a diradical selected from the group consisting of —O—, —S—, —NH— and —CH═CH— and which is optionally substituted with one or more substituent(s) selected from the group consisting of aryl, haloaryl, cyanoaryl, heteroaryl, C₃₋₁₀-cycloalkyl, aryl-C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl; or C₃₋₁₀-cycloalkyl- optionally substituted with one or more substituent(s) selected from the group consisting of aryl, haloaryl, cyanoaryl, heteroaryl, C₃₋₁₀-cycloalkyl, carboxyl, 5-tetrazolyl, acylaminosulfonyl, sulfonylaminocarbonyl, and a straight or branched C₁₋₆-alkyl,
W¹ and W² independently represent —O—, —CH₂— or —S—,
Y¹ represents —OH or —SH,
Y² represents ═O or ═S,
W³ represents a bond or a spacer, and
the term ‘molecule’ represents a fragment obtained by formal abstraction of a hydrogen atom from an amino group, a hydroxy group, or a mercapto group of a therapeutically effective polypeptide,
with the proviso that at least either A represents —(CH₂)₁₁₋₂₀— in which one or more methylene groups are optionally replaced by a diradical selected from the group consisting of —O—, —S—, —NH— and —CH═CH— and which is optionally substituted with one or more substituent(s) selected from the group consisting of C₁₋₆-alkyl, C₁₋₆-alkoxy or carboxy, or R represents C₁₁₋₂₀-alkyl-, in which one or more methylene groups are optionally replaced by a diradical selected from the group consisting of —O—, —S—, —NH— and —CH═CH— and which is optionally substituted with one or more substituent(s) selected from the group consisting of aryl, haloaryl, cyanoaryl, heteroaryl, C₃₋₁₀-cycloalkyl, aryl-C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl.

The optional spacer W³ can be a divalent molecular fragment able to covalently connect to the amino group, hydroxy group, or mercapto group of the therapeutically effective polypeptide. This divalent or polyvalent molecular fragment may also have an influence on the biological properties of the conjugate molecule-protracting tag, and structural modifications of this spacer may be used to adjust and improve the properties of the conjugate.

In one aspect of the invention, the spacer is a combination of one or several different structural elements selected from but not limited to alkylene chains, partially or fully fluorinated alkylene chains, arylenes, heteroarylenes, oligo(ethylene glycol), amide bonds, lysine, short peptides, an amino acid, short oligoamides, and other, similar fragments.

In another aspect of the invention, the spacer W³ is selected from the group consisting of oligo(ethylene glycol), an amino acid or a combination thereof.

In a further aspect of the invention, the spacer W³ is selected from the group consisting of:

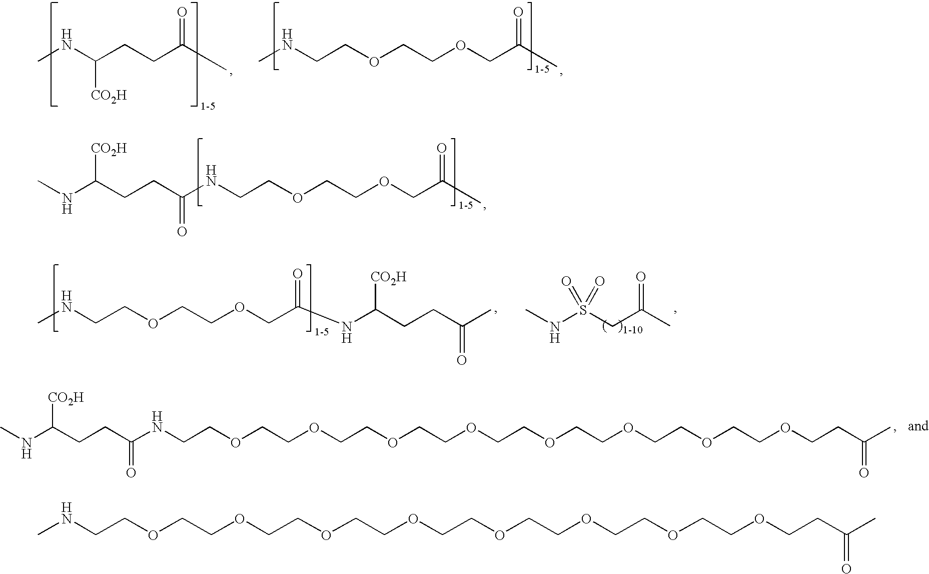

Depending on the precise therapeutic target and on the length of the optional spacer W³ the tagged therapeutically effective polypeptide may exert its activity while bound to a plasma protein, or the tagged therapeutically effective polypeptide may show a diminished biological activity while bound to the plasma protein, and only the unbound fraction of tagged therapeutically effective polypeptide display the full biological activity. All these different features are included within the scope of the present invention.

Serum albumin has a half-life of more than one week, and one approach to increasing the plasma half-life of peptides has been to derivatise the peptides with a chemical entity that binds to serum albumin.

In one aspect of the invention, a compound with formula I wherein the molecule is therapeutically active while being covalently or not covalently bound to albumin, is provided.

In another aspect of the invention, a compound with formula I wherein the plasma half-life of the compound with formula I is increased compared to the plasma half-life of a compound without the protractor of general formula II, is provided.

In another aspect of the invention, a compound with formula I wherein the pharmacological effect of the compound with formula I is prolonged compared to the pharmacological effect of a compound without the compound of general formula II, is provided.

In yet a further aspect the invention provides a protractor compound of the general formula II

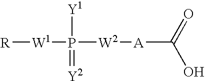

wherein
A represents —(CH₂)₁₋₂₀— in which one or more methylene groups are optionally replaced by a diradical selected from the group consisting of —O—, —S—, —NH— and —CH═CH— and which is optionally substituted with one or more substituent(s) selected from the group consisting of C₁₋₆-alkyl, C₁₋₆-alkoxy or carboxy,
R represents C₁₋₂₀-alkyl- in which one or more methylene groups are optionally replaced by a diradical selected from the group consisting of —O—, —S—, —NH— and —CH═CH— and which is optionally substituted with one or more substituent(s) selected from the group consisting of aryl, haloaryl, cyanoaryl, heteroaryl, C₃₋₁₀-cycloalkyl, aryl-C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl; or C₃₋₁₀-cycloalkyl- optionally substituted with one or more substituent(s) selected from the group consisting of aryl, haloaryl, cyanoaryl, heteroaryl, C₃₋₁₀-cycloalkyl, carboxyl, 5-tetrazolyl, acylaminosulfonyl, sulfonylaminocarbonyl, and a straight or branched C₁₋₆-alkyl,
W¹ and W² independently represent —O—, —CH₂— or —S—,
Y¹ represents —OH or —SH, and

Y² represents ═O or ═S,

with the proviso that at least either A represents —(CH₂)₁₁₋₂₀— in which one or more methylene groups are optionally replaced by a diradical selected from the group consisting of —O—, —S—, —NH— and —CH═CH— and which is optionally substituted with one or more substituent(s) selected from the group consisting of C₁₋₆-alkyl, C₁₋₆-alkoxy or carboxy, or R represents C₁₁₋₂₀-alkyl- in which one or more methylene groups are optionally replaced by a diradical selected from the group consisting of —O—, —S—, —NH— and —CH═CH— and which is optionally substituted with one or more substituent(s) selected from the group consisting of aryl, haloaryl, cyanoaryl, heteroaryl, C₃₋₁₀-cycloalkyl, aryl C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl.

In one aspect of the invention, A represents —(CH₂)₁₋₂₀— in which one or more methylene groups are optionally replaced by a diradical selected from the group consisting of —O—, —S—, —NH— and —CH═CH— and R represents C₁₁₋₂₀-alkyl- in which one or more methylene groups are optionally replaced by a diradical selected from the group consisting of —O—, —S—, —NH— and —CH═CH— and which is optionally substituted with one or more substituent(s) selected from the group consisting of aryl, haloaryl, cyanoaryl, heteroaryl, C₃₋₁₀-cycloalkyl, aryl C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl.

In another aspect of the invention, A represents —(CH₂)₁₁₋₂₀— in which one or more methylene groups are optionally replaced by a diradical selected from the group consisting of —O—, —S—, —NH— and —CH═CH— and which is optionally substituted with one or more substituent(s) selected from the group consisting of C₁₋₆-alkyl, C₁₋₆-alkoxy or carboxy.

In a further aspect of the invention, R represents C₁₋₂₀-alkyl- in which one or more methylene groups are optionally replaced by a diradical selected from the group consisting of —O—, —S—, —NH— and —CH═CH— and which is optionally substituted with one or more substituent(s) selected from the group consisting of aryl, haloaryl, cyanoaryl, heteroaryl, C₃₋₁₀-cycloalkyl, aryl-C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl.

In another aspect of the invention, A represents —(CH₂)₁₁₋₂₀— in which one or more methylene groups are optionally replaced by a diradical selected from the group consisting of —O—, —S—, —NH— and —CH═CH—.

In another aspect of the invention, A represents —(CH₂)₁₁₋₂₀—.

In another aspect of the invention, A represents —(CH₂)₁₁₋₁₈—.

In another aspect of the invention, R represents C₁₋₂₀-alkyl- in which one or more methylene groups are optionally replaced by a diradical selected from the group consisting of —O—, —S—, —NH— and —CH═CH—.

In another aspect of the invention, R represents C₁₋₂₀-alkyl-.

In another aspect of the invention, R represents C₁₁₋₂₀-alkyl-.

In another aspect of the invention, R represents C₁₄₋₁₈-alkyl-.

In another aspect of the invention, A represents —(CH₂)₁₋₂₀— in which one or more methylene groups are replaced by —O—.

In another aspect of the invention, A represents —(CH₂)₁₋₂₀— in which one or more methylene groups are replaced by —S—.

In another aspect of the invention, A represents —(CH₂)₁₋₂₀— in which one or more methylene groups are replaced by —CH═CH—.

In another aspect of the invention, A represents —(CH₂)₁₁₋₂₀— in which three methylene groups are replaced.

In another aspect of the invention, W¹ represents —O—.

In another aspect of the invention, W² represents —O— or —CH₂—.

In another aspect of the invention, Y¹ represents —OH.

In another aspect of the invention, Y² represents ═O.

In another aspect of the invention, the molecule is a fragment obtained via formal abstraction of a hydrogen atom of an amino group of a therapeutically effective polypeptide.

In another aspect of the invention, the molecule is a fragment obtained via formal abstraction of a hydrogen atom of the N-terminal amino group of a therapeutically effective polypeptide.

In another aspect of the invention, the molecule is a fragment obtained via formal abstraction of a hydrogen atom of the ε-amino group of a lysine residue of a therapeutically effective polypeptide.

In another aspect of the invention, the molecule is a fragment obtained via formal abstraction of a hydrogen atom of the thiol group of a cysteine residue.

In another aspect the present invention provides a compound according to formula (I), wherein the polypeptide is an insulinotropic peptide.

In one embodiment the invention provides a compound according to formula (I), wherein the polypeptide is GLP-1(7-37) or a variant thereof.

In another embodiment the invention provides a compound according to formula (I), wherein the polypeptide is GLP-1(7-37) or an analog thereof.

In another embodiment the invention provides a compound according to formula (I), wherein the polypeptide comprises the amino acid sequence of the formula (III):

Formula (III)

(SEQ ID No: 3)

Xaa₇-Xaa₈-Glu-Gly-Thr-Phe-Thr-Ser-Asp-Xaa₁₆-Ser-
Xaa₁₈-Xaa₁₉-Xaa₂₀-Glu-Xaa₂₂-Xaa₂₃-Ala-Xaa₂₅-Xaa₂₆-
Xaa₂₇-Phe-Ile-Xaa₃₀-Trp-Leu-Xaa₃₃-Xaa₃₄-Xaa₃₅-
Xaa₃₆-Xaa₃₇-Xaa₃₈-Xaa₃₉-Xaa₄₀-Xaa₄₁-Xaa₄₂-Xaa₄₃-
Xaa₄₄-Xaa₄₅-Xaa₄₆

wherein Xaa₇ is L-histidine, D-histidine, desamino-histidine, 2-amino-3-(2-aminoimidazol-4-yl)propionic acid, β-hydroxy-histidine, homohistidine, N^{α}-acetyl-histidine, α-fluoromethyl-histidine, α-methyl-histidine, 3-pyridylalanine, 2-pyridylalanine or 4-pyridylalanine;
Xaa₈ is Ala, Gly, Val, Leu, Ile, Lys, Aib, 1-aminocyclopropanecarboxylic acid, 1-aminocyclobutanecarboxylic acid, 1-aminocyclopentanecarboxylic acid, 1-aminocyclohexanecarboxylic acid, 1-aminocycloheptanecarboxylic acid, or 1-aminocyclooctanecarboxylic acid;

### Xaa₁₆ is Val or Leu;

### Xaa₁₈ is Ser, Lys or Arg;

### Xaa₁₉ is Tyr or Gln;

### Xaa₂₀ is Leu or Met;

### Xaa₂₂ is Gly, Glu or Aib;

### Xaa₂₃ is Gln, Glu, Lys or Arg;

### Xaa₂₅ is Ala or Val;

### Xaa₂₆ is Lys, Glu or Arg;

### Xaa₂₇ is Glu or Leu;

### Xaa₃₀ is Ala, Glu or Arg;

### Xaa₃₃ is Val or Lys;

### Xaa₃₄ is Lys, Glu, Asn or Arg;

### Xaa₃₅ is Gly or Aib;

### Xaa₃₆ is Arg, Gly or Lys;

Xaa₃₇ is Gly, Ala, Glu, Pro, Lys, amide or is absent;
Xaa₃₈ is Lys, Ser, amide or is absent;
Xaa₃₉ is Ser, Lys, amide or is absent;
Xaa₄₀ is Gly, amide or is absent;
Xaa₄₁ is Ala, amide or is absent;
Xaa₄₂ is Pro, amide or is absent;
Xaa₄₃ is Pro, amide or is absent;
Xaa₄₄ is Pro, amide or is absent;
Xaa₄₅ is Ser, amide or is absent;
Xaa₄₆ is amide or is absent;
provided that if Xaa₃₈, Xaa₃₉, Xaa₄₀, Xaa₄₁, Xaa₄₂, Xaa₄₃, Xaa₄₄, Xaa₄₅ or Xaa₄₆ is absent then each amino acid residue downstream is also absent.

In another aspect the invention provides a compound according to formula (I), wherein the polypeptide comprises the amino acid sequence of formula (IV):

Formula (IV)

(SEQ ID No: 4)

Xaa₇-Xaa₈-Glu-Gly-Thr-Phe-Thr-Ser-Asp-Val-Ser-
Xaa₁₈-Tyr-Leu-Glu-Xaa₂₂-Xaa₂₃-Ala-Ala-Xaa₂₆-Glu-
Phe-Ile-Xaa₃₀-Trp-Leu-Val-Xaa₃₄-Xaa₃₅-Xaa₃₆-Xaa₃₇-
Xaa₃₈

wherein
Xaa₇ is L-histidine, D-histidine, desamino-histidine, 2-aminohistidine, β-hydroxy-histidine, homohistidine, N^{α}-acetyl-histidine, α-fluoromethyl-histidine, α-methyl-histidine, 3-pyridylalanine, 2-pyridylalanine or 4-pyridylalanine;
Xaa₈ is Ala, Gly, Val, Leu, Ile, Lys, Aib, 1-aminocyclopropanecarboxylic acid, 1-aminocyclobutanecarboxylic acid, 1-aminocyclopentanecarboxylic acid, 1-aminocyclohexanecarboxylic acid, 1-aminocycloheptanecarboxylic acid, or 1-aminocyclooctanecarboxylic acid;

### Xaa₁₈ is Ser, Lys or Arg;

### Xaa₂₂ is Gly, Glu or Aib;

### Xaa₂₃ is Gln, Glu, Lys or Arg;

### Xaa₂₆ is Lys, Glu or Arg;

### Xaa₃₀ is Ala, Glu or Arg;

### Xaa₃₄ is Lys, Glu or Arg;

### Xaa₃₅ is Gly or Aib;

### Xaa₃₆ is Arg or Lys;

### Xaa₃₇ is Gly, Ala, Glu or Lys;

Xaa₃₈ is Lys, amide or is absent.

In another aspect the invention provides a compound according to formula (I), wherein the polypeptide is selected from GLP-1(7-35), GLP-1(7-36), GLP-1(7-36)-amide, GLP-1(7-37), GLP-1(7-38), GLP-1(7-39), GLP-1(7-40), GLP-1(7-41) or an analog thereof.

In another aspect the invention provides a compound according to formula (I), wherein the polypeptide comprises no more than fifteen amino acid residues which have been exchanged, added or deleted as compared to GLP-1(7-37) (SEQ ID No. 1), or no more than ten amino acid residues which have been exchanged, added or deleted as compared to GLP-1(7-37) (SEQ ID No. 1).

In another aspect the invention provides a compound according to formula (I), wherein the polypeptide comprises no more than six amino acid residues which have been exchanged, added or deleted as compared to GLP-1(7-37) (SEQ ID No. 1).

In another aspect the invention provides a compound according to formula (I), wherein the molecule is a polypeptide comprising no more than 4 amino acid residues which are not encoded by the genetic code.

In another aspect the invention provides a compound according to formula (I), wherein the polypeptide is a DPP-IV protected insulinotropic peptide.

In another aspect the invention provides a compound according to formula (I), wherein the polypeptide comprises an Aib residue in position 8.

In another aspect the invention provides a compound according to formula (I), wherein the polypeptide is a GLP-1(7-37) analog wherein the amino acid residue in position 7 of said polypeptide is selected from the group consisting of D-histidine, desamino-histidine, 2-amino-3-(2-aminoimidazol-4-yl)propionic acid, β-hydroxy-histidine, homohistidine, N^{α}-acetyl-histidine, α-fluoromethyl-histidine, α-methyl-histidine, 3-pyridylalanine, 2-pyridylalanine and 4-pyridylalanine.

In another aspect the invention provides a compound according to formula (I), wherein the polypeptide is a GLP-1(7-37) analog selected from the group consisting of Arg³⁴GLP-1(7-37), Lys³⁸Arg^{26,34}GLP-1(7-38), Lys³⁸Arg^{26,34}GLP-1(7-38)-OH, Lys³⁶Arg^{26,34}GLP-1(7-36),

### Aib^{8,22,35}GLP-1(7-37), Aib^{8,35}GLP-1(7-37), Aib^{8,22}GLP-1(7-37),

### Aib^{8,22,35}Arg^{26,34}Lys³⁸GLP-1(7-38), Aib^{8,35}Arg^{26,34}Lys³⁸GLP-1(7-38),

### Aib^{8,22}Arg^{26,34}Lys³⁸GLP-1(7-38), Aib^{8,22,35}Arg^{26,34}Lys³⁸GLP-1(7-38),

### Aib^{8,35}Arg^{26,34}Lys³⁸GLP-1(7-38), Aib^{8,22,35}Arg²⁶Lys³⁸GLP-1(7-38),

### Aib^{8,35}Arg²⁶Lys³⁸GLP-1(7-38), Aib^{8,22}Arg²⁶Lys³⁸GLP-1(7-38),

Aib^{8,22,35}Arg³⁴ Lys³⁸GLP-1(7-38), Aib^{8,35}Arg³⁴Lys³⁸GLP-1(7-38), Aib^{8,22}Arg³⁴Lys³⁸GLP-1(7-38),
Aib^{8,22,35}Ala³⁷Lys³⁸GLP-1(7-38), Aib^{8,35}Ala³⁷Lys³⁸GLP-1(7-38), Aib^{8,22}Ala³⁷Lys³⁸GLP-1(7-38),

### Aib^{8,22,35}Lys³⁷GLP-1(7-37), Aib^{8,35}Lys³⁷GLP-1(7-37) and Aib^{8,22}Lys³⁷GLP-1(7-38).

In another aspect the invention provides a compound according to formula (I), wherein the polypeptide is GLP-1(7-37) or an analog thereof which is attached via a bond or an optional spacer to the protractor via the amino acid residue in position 23, 26, 34, 36 or 38 relative to the amino acid sequence SEQ ID No:1.

In another aspect the present invention provides a compound according to formula (I),

wherein the polypeptide is exendin-4(1-39) or an analog thereof.

In one aspect the invention provides a compound according to formula (I), wherein the polypeptide is an exendin-4 analog comprising no more than twelve amino acid residues which have been exchanged, added or deleted as compared to exendin-4(1-39) (SEQ ID No. 2), or no more than eight amino acid residues which have been exchanged, added or deleted as compared to exendin-4(1-39) (SEQ ID No. 2).

In another aspect the invention provides a compound according to formula (I), wherein the polypeptide is ZP-10, i.e. HGEGTFTSDLSKQMEEEAVRLFIEWLKNGGPSSGAPPSKKKKKK-amide (SEQ ID No. 5).

One aspect of the invention, relates to a compound according to formula (I) wherein the polypeptide is a polypeptide that binds to the MC4 receptor. Examples of such polypeptides are described in WO 2004/099246 which is incorporated by reference.

Another aspect of the invention, relates to a compound of formula I wherein the polypeptide is a polypeptide with formula V

R¹-X-X¹-X²-X³-X⁴-X⁵-X⁶-X⁷-X⁸-X⁹-X¹⁰-X¹¹-R² [V]

wherein R¹, which is bonded to an N-terminal amino group, is either absent or represents
C₁₋₄alkanoyl;
X represents a bond or an amino acid, a di- or tri-peptide residue, wherein the amino acid(s) may be natural or synthetic;
X¹ represents a bond or an amino acid residue with a functional group in the side chain to which a protracting group may be attached;
X² represents a bond or an amino acid, di-, tri- or tetra-peptide residue, wherein the amino acid(s) may be natural or synthetic;
X³ represents a bond or an amino acid residue optionally capable of making a bridge to X¹⁰;
X⁴ represents a bond or an amino acid or di-peptide residue, wherein the amino acid(s) may be natural or synthetic;
X⁵ represents an amino acid residue selected from His, Ala, Nle, Met, Met(O), Met(O₂), Gln, Gln(ε-alkyl), Gln(ε-aryl), Asn, Asn(ε-alkyl), Asn(ε-aryl), Ser, Thr, Cys, F-Pro, Pro, Hyp, (S)-1,2,3,4-tetrahydroisoquinoline-3-carboxylic acid, Trp, 1-naphthylalanine, 2-naphthylalanine, 2-pyridylalanine, 3-pyridylalanine, 4-pyridylalanine, 2-thienylalanine, 3-thienylalanine, 4-thiazolylalanine, 2-furylalanine, 3-furylalanine, Phe, wherein the phenyl moiety of said Phe is optionally substituted by halogen, hydroxyl, alkoxy, nitro, benzoyl, methyl, trifluoromethyl or cyano;
X⁶ represents (D)-Phe, wherein the phenyl moiety of said (D)-Phe is optionally substituted with halogen, hydroxy, alkoxy, nitro, methyl, trifluoromethyl or cyano;
X⁷ represents Arg;
X⁸ represents Trp or 2-naphtylalanine;
X⁹ represents a bond or an amino acid, or di-peptide residue, wherein the amino acid(s) may be natural or synthetic;
X¹⁰ represents a bond or an amino acid residue optionally capable of making a bridge to X³;
X¹¹ represents a bond, an amino acid or a di-peptide, wherein the amino acid(s) may be natural or synthetic;
R² represents —OH or —NRR′, wherein R and R′ independently represent hydrogen, C₁₋₈alkyl, C₂₋₈alkenyl or C₂₋₈alkynyl;
wherein the peptide of formula I is optionally cyclized from X³ to X¹⁰ via a lactame or a disulfide bridge;
with the proviso that the polypeptide of formula V comprises at least 7 amino acid residues;
and any pharmaceutically acceptable salt, solvate or hydrate thereof.

In one aspect of the invention, the molecule in the compound with formula I is obtained by formal extraction of a hydrogen atom from an amino group of a polypeptide of formula V.

In one aspect of the invention, the molecule in the compound with formula I is obtained by formal extraction of a hydrogen atom from the N-terminal amino group, in which case R1 is absent of a polypeptide of formula V.

In another aspect of the invention, the molecule in the compound with formula I is obtained by formal extraction of a hydrogen atom from an amino group of the sidechain of X¹ of a polypeptide of formula V.

Another aspect of the invention, relates to a compound according to formula (I) wherein the polypeptide is a polypeptide of formula V wherein there is a bond between X³ and X¹⁰ to make the compound of formula V cyclic by a disulfide bridge (X³, X¹⁰ are independently Cys or homoCys) or by an lactam bond between an acid in the side chain of X³ or X¹⁰ and an amine in the side chain of X¹⁰ or X³.

Another aspect of the invention, relates to a compound according to formula (I) wherein the polypeptide is a polypeptide of formula V wherein X is a bond.

Another aspect of the invention, relates to a compound according to formula (I) wherein the polypeptide is a polypeptide of formula V wherein X¹ represents a bond.

Another aspect of the invention, relates to a compound according to formula (I) wherein the polypeptide is a polypeptide of formula V wherein X² represents Nle.

Another aspect of the invention, relates to a compound according to formula (I) wherein the polypeptide is a polypeptide of formula V wherein X³ represents Glu or Aps and X¹⁰ represents Lys, Orn, 2,4-diamino butyric acid or 2,3-diamino propionic acid.

Another aspect of the invention, relates to a compound according to formula (I) wherein the polypeptide is a polypeptide of formula V wherein X³ represents Glu or Asp, and X¹⁰ represents Lys.

Another aspect of the invention, relates to a compound according to formula (I) wherein the polypeptide is a polypeptide of formula V wherein X⁴ represents a bond.

Another aspect of the invention, relates to a compound according to formula (I) wherein the polypeptide is a polypeptide of formula V wherein X⁵ represents Ala, Nle, Met, Met(O), Met(O₂), Gln, Gln(ε-alkyl), Gln(ε-aryl), Asn, Asn (ε-alkyl), Asn(ε-aryl), Ser, Thr, Cys, F-Pro, Pro, Hyp, (S)-1,2,3,4-tetrahydroisoquinoline-3-carboxylic acid, Trp, 1-naphthylalanine, 2-naphthylalanine, 2-PyAla, 3-PyAla, 4-PyAla, 2-thienylalanine, 3-thienylalanine, 4-thiazolylalanine, 2-furylalanine, 3-furylalanine, Phe, wherein the phenyl moiety of said Phe is optionally substituted by halogen, hydroxyl, alkoxy, nitro, benzoyl, methyl, trifluoromethyl or cyano.

Another aspect of the invention, relates to a compound according to formula (I) wherein the polypeptide is a polypeptide of formula V wherein X⁵ represents His.

Another aspect of the invention, relates to a compound according to formula (I) wherein the polypeptide is a polypeptide of formula V wherein X⁵ represents 3-PyAla, Hyp, Gln or Asn.

Another aspect of the invention, relates to a compound according to formula (I) wherein the polypeptide is a polypeptide of formula V wherein X⁹ represents a bond.

Another aspect of the invention, relates to a compound according to formula (I) wherein the polypeptide is a polypeptide of formula V wherein X¹¹ represents a bond.

Another aspect of the invention, relates to a compound according to formula (I) wherein the polypeptide is a polypeptide of formula V wherein R² represents —NH₂.

Another aspect of the invention, relates to a compound according to formula (I) wherein the polypeptide is a polypeptide of formula V wherein X⁶-X⁷-X⁸-X⁹-X¹⁰ represents D-Phe-Arg-Trp-Lys.

Another aspect of the invention, relates to a compound according to formula (I) wherein the polypeptide is a polypeptide of formula V selected from amongst

### H-Nle-c[Asp-3-PyAla-D-Phe-Arg-Trp-Lys]-R²;

### H-Nle-c[Glu-Hyp-D-Phe-Arg-Trp-Lys]-R²;

### H-Nle-c[Asp-Hyp-D-Phe-Arg-Trp-Lys]-R²;

### H-Nle-c[Glu-Gln-D-Phe-Arg-Trp-Lys]-R²;

### H-Nle-c[Asp-Gln-D-Phe-Arg-Trp-Lys]-R²;

### H-Nle-c[Glu-Asn-D-Phe-Arg-Trp-Lys]-R²;

### H-Nle-c[Asp-Asn-D-Phe-Arg-Trp-Lys]-R²;

### H-Nle-c[Glu-3-PyAla-D-Phe-Arg-2NaI-Lys]-R²;

### H-Nle-c[Asp-3-PyAla-D-Phe-Arg-2NaI-Lys]-R²;

### H-Nle-c[Glu-Hyp-D-Phe-Arg-2NaI-Lys]-R²;

### H-Nle-c[Asp-Hyp-D-Phe-Arg-2NaI-Lys]-R²;

### H-Nle-c[Glu-Gln-D-Phe-Arg-2NaI-Lys]-R²;

### H-Nle-c[Asp-Gln-D-Phe-Arg-2NaI-Lys]-R²;

### H-Nle-c[Glu-Asn-D-Phe-Arg-2NaI-Lys]-R²; and

### H-Nle-c[Asp-Asn-D-Phe-Arg-2NaI-Lys]-R².

Another aspect of the invention, relates to a compound according to formula (I) wherein the polypeptide is a polypeptide of formula V wherein X-X¹-X²-X³-X⁴-X⁵-X⁶-X⁷-X⁸-X⁹-X¹⁰-R² represents Nle-c[Glu-3-PyAla-D-Phe-Arg-Trp-Lys]-NH₂ or Nle-c[Glu-Hyp-D-Phe-Arg-Trp-Lys]-NH₂.

Another aspect of the invention, relates to a compound according to formula (I) wherein the polypeptide is a polypeptide of formula V wherein X-X¹-X²-X³-X⁴-X⁵-X⁶-X⁷-X⁸-X⁹-X¹⁰-R² represents Nle-c[Glu-His-D-Phe-Arg-Trp-Lys]-NH₂.

Another aspect of the invention, relates to a compound according to formula (I) wherein the polypeptide is a polypeptide of formula V wherein X-X¹-X² is represented by a moiety of the formula Z¹-Z²-Z³-Z⁴-Z⁵-Z⁶, wherein

Z¹ represents Gly;
Z² represents Ser, (D)-Ser or Thr;
Z³ represents Gln, Asn, (D)-Gln or (D)-Asn;
Z⁴ represents His, homoArg, Arg, Lys or Orn;
Z⁵ represents Ser, (D)-Ser or Thr; and
Z⁶ represents Nle.

Another aspect of the invention, relates to a compound according to formula (I) wherein the polypeptide is a polypeptide of formula V wherein X³ represents Glu, and X¹⁰ represents Lys.

Another aspect of the invention, relates to a compound according to formula (I) wherein the polypeptide is a polypeptide of formula V wherein X⁴, X⁹ and X¹¹ represent a bond.

Another aspect of the invention, relates to a compound according to formula (I) wherein the polypeptide is a polypeptide of formula V wherein X⁵ represents Ala, Nle, Met, Met(O), Met(O2), Gln, Gln(ε-alkyl), Gln(ε-aryl), Asn, Asn (ε-alkyl), Asn(ε-aryl), Ser, Thr, Cys, Pro, F-Pro, Hyp, (S)-1,2,3,4-tetrahydroisoquinoline-3-carboxylic acid, Trp, 1-naphthylalanine, 2-naphthylalanine, 2-PyAla, 3-PyAla, 4-PyAla, 2-thienylalanine, 3-thienylalanine, 4-thiazolylalanine, 2-furylalanine, 3-furylalanine, Phe, wherein the phenyl moiety of said Phe is optionally substituted by halogen, hydroxyl, alkoxy, nitro, benzoyl, methyl, trifluoromethyl or cyano.

Another aspect of the invention, relates to a compound according to formula (I) wherein the polypeptide is a polypeptide of formula V wherein X⁵ represents F-Pro, Hyp or Gln.

Another aspect of the invention, relates to a compound according to formula (I) wherein the polypeptide is a polypeptide of formula V wherein R² represents —NH₂.

Another aspect of the invention, relates to a compound according to formula (I) wherein the polypeptide is a polypeptide of formula V wherein X⁶-X⁷-X⁸-X⁹-X¹⁰ represents (D)-Phe-Arg-Trp-Lys.

Another aspect of the invention, relates to a compound according to formula (I) wherein the polypeptide is a polypeptide of formula V wherein the moiety of the formula Z¹-Z²-Z³-Z⁴-Z⁵-Z⁶ is selected from amongst

### Gly-Ser-Asn-Asn-Thr-Nle;

### Gly-Ser-Asn-homoArg-Thr-Nle;

### Gly-Ser-DAsn-His-Thr-Nle;

### Gly-Ser-DAsn-homoArg-Thr-Nle;

### Gly-Ser-Gln-Arg-Ser-Nle;

### Gly-Ser-Gln-His-Ser-Nle;

### Gly-Ser-Gln-homoArg-Ser-Nle;

### Gly-Ser-Gln-homoArg-Thr-Nle;

### Gly-Ser-Gln-Lys-Ser-Nle;

### Gly-Ser-Gln-Orn-Ser-Nle;

### Gly-Ser-Ser-His-Thr-Nle and

### Gly-Ser-Ser-Tyr-Thr-Nle.

Another aspect of the invention, relates to a compound according to formula (I) wherein the polypeptide is a polypeptide of formula V wherein X³-X⁴-X⁵-X⁶-X⁷-X⁸-X⁹-X¹⁰-X¹¹-R² is selected from

cyclo[Glu-3-PyAla-(D)-Phe-Arg-Trp-Lys]-NH₂;
cyclo[Glu-F-Pro-(D)-Phe-Arg-Trp-Lys]-NH₂;
cyclo[Glu-Gln-(D)-Phe-Arg-Trp-Lys]-NH₂;
cyclo[Glu-Hyp-(D)-Phe-Arg-Trp-Lys]-NH₂; and
cyclo[Glu-Met(O2)-(D)-Phe-Arg-Trp-Lys]-NH₂.

Another aspect of the invention, relates to a compound according to formula (I) wherein the polypeptide is a polypeptide of formula V wherein the compound of formula V is non-cyclic.

Another aspect of the invention, relates to a compound according to formula (I) wherein the polypeptide is a non-cyclic polypeptide of formula V wherein X represents a bond.

Another aspect of the invention, relates to a compound according to formula (I) wherein the polypeptide is a non-cyclic polypeptide of formula V wherein X represents an amino acid residue.

Another aspect of the invention, relates to a compound according to formula (I) wherein the polypeptide is a non-cyclic polypeptide of formula V wherein X represents Ser.

Another aspect of the invention, relates to a compound according to formula (I) wherein the polypeptide is a non-cyclic polypeptide of formula V wherein X¹ represents Lys.

Another aspect of the invention, relates to a compound according to formula (I) wherein the polypeptide is a non-cyclic polypeptide of formula V wherein X¹ represents a bond.

Another aspect of the invention, relates to a compound according to formula (I) wherein the polypeptide is a non-cyclic polypeptide of formula V wherein X² represents Tyr-Ser-Nle.

Another aspect of the invention, relates to a compound according to formula (I) wherein the polypeptide is a non-cyclic polypeptide of formula V wherein X² represents Ser-Nle.

Another aspect of the invention, relates to a compound according to formula (I) wherein the polypeptide is a non-cyclic polypeptide of formula V wherein X² represents Ser-Tyr-Ser-Nle.

Another aspect of the invention, relates to a compound according to formula (I) wherein the polypeptide is a non-cyclic polypeptide of formula V wherein X³ represents Glu.

Another aspect of the invention, relates to a compound according to formula (I) wherein the polypeptide is a non-cyclic polypeptide of formula V wherein X⁴ represents a bond.

Another aspect of the invention, relates to a compound according to formula (I) wherein the polypeptide is a non-cyclic polypeptide of formula V wherein X⁵ represents Ala, Nle, Met, Met(O), Met(O₂), Gln, Gln(ε-alkyl), Gln(ε-aryl), Asn, Asn (ε-alkyl), Asn(ε-aryl), Ser, Thr, Cys, F-Pro, Pro, Hyp, (S)-1,2,3,4-tetrahydroisoquinoline-3-carboxylic acid, Trp, 1-naphthylalanine, 2-naphthylalanine, 2-PyAla, 3-PyAla, 4-PyAla, 2-thienylalanine, 3-thienylalanine, 4-thiazolylalanine, 2-furylalanine, 3-furylalanine, Phe, wherein the phenyl moiety of said Phe is optionally substituted by halogen, hydroxyl, alkoxy, nitro, benzoyl, methyl, trifluoromethyl or cyano.

Another aspect of the invention, relates to a compound according to formula (I) wherein the polypeptide is a non-cyclic polypeptide of formula V wherein X⁵ represents His.

Another aspect of the invention, relates to a compound according to formula (I) wherein the polypeptide is a non-cyclic polypeptide of formula V wherein X⁵ represents Gln, Hyp, 3-PyAla, Ala or Ser.

Another aspect of the invention, relates to a compound according to formula (I) wherein the polypeptide is a non-cyclic polypeptide of formula V wherein X⁹ represents Gly.

Another aspect of the invention, relates to a compound according to formula (I) wherein the polypeptide is a non-cyclic polypeptide of formula V wherein X¹⁰ represents Lys or Arg.

Another aspect of the invention, relates to a compound according to formula (I) wherein the polypeptide is a non-cyclic polypeptide of formula V wherein X¹¹ represents Pro-Val.

Another aspect of the invention, relates to a compound according to formula (I) wherein the polypeptide is a non-cyclic polypeptide of formula V wherein R² represents —NH₂.

Another aspect of the invention, relates to a compound according to formula (I) wherein the polypeptide is a polypeptide of formula V wherein R¹-X-X¹-X²-X³-X⁴-X⁵-X⁶-X⁷-X⁸-X⁹-X¹⁰-R² represents a compound selected from amongst

### CH₃C(O)-Lys-Tyr-Ser-Nle-Glu-Hyp-D-Phe-Arg-Trp-Gly-Lys-Pro-Val-NH₂,

### CH₃C(O)-Lys-Tyr-Ser-Nle-Glu-3-PyAla-D-Phe-Arg-Trp-Gly-Lys-Pro-Val-NH₂,

### CH₃C(O)-Lys-Tyr-Ser-Nle-Glu-Ser-D-Phe-Arg-Trp-Gly-Arg-Pro-Val-NH₂,

### CH₃C(O)-Lys-Tyr-Ser-Nle-Glu-Ala-D-Phe-Arg-Trp-Gly-Arg-Pro-Val-NH₂,

### CH₃C(O)-Lys-Tyr-Ser-Nle-Glu-Gln-D-Phe-Arg-Trp-Gly-Arg-Pro-Val-NH₂,

### CH₃C(O)-Lys-Tyr-Ser-Nle-Glu-Hyp-D-Phe-Arg-Trp-Gly-Arg-Pro-Val-NH₂.

Another aspect of the invention, relates to a compound according to formula (I) wherein the polypeptide is a polypeptide of formula V wherein R¹-X-X¹-X²-X³-X⁴-X⁵-X⁶-X⁷-X⁸-X⁹-X¹⁰-X¹¹-R² represents a compound selected from amongst

### CH₃C(O)-Ser-Lys-Ser-Nle-Glu-Hyp-D-Phe-Arg-Trp-Gly-Lys-Pro-Val-NH₂,

### CH₃C(O)-Ser-Lys-Ser-Nle-Glu-3-PyAla-D-Phe-Arg-Trp-Gly-Lys-Pro-Val-NH₂,

### CH₃C(O)-Ser-Lys-Ser-Nle-Glu-Ser-D-Phe-Arg-Trp-Gly-Arg-Pro-Val-NH₂,

### CH₃C(O)-Ser-Lys-Ser-Nle-Glu-Ala-D-Phe-Arg-Trp-Gly-Arg-Pro-Val-NH₂,

### CH₃C(O)-Ser-Lys-Ser-Nle-Glu-Gln-D-Phe-Arg-Trp-Gly-Arg-Pro-Val-NH₂,

### CH₃C(O)-Ser-Lys-Ser-Nle-Glu-Hyp-D-Phe-Arg-Trp-Gly-Arg-Pro-Val-NH₂,

### CH₃C(O)-Ser-Lys-Ser-Nle-Glu-3-PyAla-D-Phe-Arg-Trp-Gly-Arg-Pro-Val-NH₂.

Another aspect of the invention, relates to a compound according to formula (I) wherein the polypeptide is a polypeptide of formula V wherein R¹-X-X¹-X²-X³-X⁴-X⁵-X⁶-X⁷-X⁸-X⁹-X¹⁰-X¹¹-R² represents a compound selected from amongst

### H-Ser-Tyr-Ser-Nle-Glu-Gln-D-Phe-Arg-Trp-Gly-Lys-Pro-Val-NH₂,

### H-Ser-Tyr-Ser-Nle-Glu-Hyp-D-Phe-Arg-Trp-Gly-Lys-Pro-Val-NH₂,

### H-Ser-Tyr-Ser-Nle-Glu-3-PyAla-D-Phe-Arg-Trp-Gly-Lys-Pro-Val-NH₂,

### H-Ser-Tyr-Ser-Nle-Glu-Ser-D-Phe-Arg-Trp-Gly-Arg-Pro-Val-NH₂,

### H-Ser-Tyr-Ser-Nle-Glu-Ala-D-Phe-Arg-Trp-Gly-Arg-Pro-Val-NH₂,

### H-Ser-Tyr-Ser-Nle-Glu-Gln-D-Phe-Arg-Trp-Gly-Arg-Pro-Val-NH₂,

### H-Ser-Tyr-Ser-Nle-Glu-Hyp-D-Phe-Arg-Trp-Gly-Arg-Pro-Val-NH₂,

### H-Ser-Tyr-Ser-Nle-Glu-3-PyAla-D-Phe-Arg-Trp-Gly-Arg-Pro-Val-NH₂.

Another aspect of the invention, relates to a compound according to formula (I) wherein the polypeptide is a polypeptide of formula V selected from the group consisting of

### H-Nle-c[Glu-His-D-Phe-Arg-Trp-Lys]-NH₂,

### H-Nle-c[Glu-3-PyAla-D-Phe-Arg-Trp-Lys]-NH₂,

### Acetyl-Ser-Lys-Ser-Nle-Glu-Ser-D-Phe-Arg-Trp-Gly-Lys-Pro-Val-NH₂,

### H-Ser-Tyr-Ser-Nle-Glu-Ser-D-Phe-Arg-Trp-Gly-Lys-Pro-Val-NH₂,

### H-Ser-Tyr-Ser-Nle-Glu-3-PyAla-D-Phe-Arg-Trp-Gly-Lys-Pro-Val-NH₂,

### H-βAla-Tyr-Ser-Nle-c[Glu-Hyp-D-Phe-Arg-Trp-Lys]-NH₂,

### H-βAla-Ala-Tyr-Ser-Nle-c[Glu-Hyp-D-Phe-Arg-Trp-Lys]-NH₂,

### Acetyl-Ser-Lys-Ser-Nle-Glu-Hyp-D-Phe-Arg-Trp-Gly-Lys-Pro-Val-NH₂,

### H-βAla-Tyr-Ser-Nle-c[Glu-Hyp-D-Phe-Arg-Trp-Lys]-NH₂,

### H-βAla-Ala-Tyr-Ser-Nle-c[Glu-Hyp-D-Phe-Arg-Trp-Lys]-NH₂,

### Acetyl-Ser-Lys-Ser-Phe-Glu-Hyp-D-Phe-Arg-Trp-Gly-Lys-Pro-Val-NH₂,

### H-Ser-Gln-Ser-Nle-c[Glu-Hyp-D-Phe-Arg-Trp-Lys]-NH₂,

### H-Gly-Ser-Gln-His-Ser-Nle-c[Glu-Hyp-D-Phe-Arg-Trp-Lys]-NH₂,

### H-Nle-c[Glu-Hyp-D-Phe-Arg-Trp-Lys]-NH₂,

### H-Gly-Ser-Gln-homoArg-Ser-Nle-c[Glu-Hyp-D-Phe-Arg-Trp-Lys]-NH₂,

### H-Gly-Ser-Gln-His-Ser-Nle-c[Glu-Met(O2)-D-Phe-Arg-Trp-Lys]-NH₂,

### H-Gly-Ser-Gln-Arg-Ser-Nle-c[Glu-Met(O2)-D-Phe-Arg-Trp-Lys]-NH₂,

### H-Gly-Ser-Gln-homoArg-Ser-Nle-c[Glu-3-PyAla-D-Phe-Arg-Trp-Lys]-NH₂,

### H-Gly-Ser-Gln-Arg-Ser-Nle-c[Glu-Met(O2)-D-Phe-Arg-Trp-Lys]-NH₂,

### H-Gly-Ser-Gln-Arg-Ser-Nle-c[Glu-Gln-D-Phe-Arg-Trp-Lys]-NH₂,

### H-Gly-Ser-Gln-His-Ser-Nle-c[Glu-Hyp-D-Phe-Arg-Trp-Lys]-NH₂,

### H-Gly-Thr-Gln-His-Ser-Nle-c[Glu-Hyp-D-Phe-Arg-Trp-Lys]-NH₂,

### H-Gly-Gln-Gln-His-Ser-Nle-c[Glu-Hyp-D-Phe-Arg-Trp-Lys]-NH₂,

### H-Gly-Thr-Gln-His-Ser-Nle-c[Glu-Asn-D-Phe-Arg-Trp-Lys]-NH₂,

### H-Gly-Glu-Thr-Gln-His-Ser-Nle-c[Glu-Hyp-D-Phe-Arg-Trp-Lys]-NH₂,

### H-Glu-Gly-Thr-Gln-His-Ser-Nle-c[Glu-Hyp-D-Phe-Arg-Trp-Lys]-NH₂),

### H-Glu-4-Abu-Thr-Gln-His-Ser-Nle-c[Glu-Hyp-D-Phe-Arg-Trp-Lys]-NH₂,

### H-His-Ser-Nle-c[Glu-Hyp-D-Phe-Arg-Trp-Lys]-NH₂,

### H-Arg-Ser-Nle-c[Glu-Hyp-D-Phe-Arg-Trp-Lys]-NH₂,

### H-Gln-Ser-Nle-c[Glu-Hyp-D-Phe-Arg-Trp-Lys]-NH₂,

### H-Glu-Ser-Nle-c[Glu-Hyp-D-Phe-Arg-Trp-Lys]-NH₂,

### H-Nle-c[Glu-Hyp-D-Phe-Arg-Trp-Lys]-NH₂,

### H-Gly-Thr-Asn-Asn-Thr-Nle-c[Glu-Hyp-D-Phe-Arg-Trp-Lys]-NH₂,

### H-Gly-Ser-D-Gln-His-Thr-Nle-c[Glu-Hyp-D-Phe-Arg-Trp-Lys]-NH₂,

### H-Gly-Ser-Gln-His-Ser-Nle-c[Glu-F-Pro-D-Phe-Arg-Trp-Lys]-NH₂,

### H-Gly-Ser-Gln-homoArg-Ser-Nle-c[Glu-F-Pro-D-Phe-Arg-Trp-Lys]-NH₂,

### H-Gly-Ser-Gln-homoArg-Thr-Nle-c[Glu-F-Pro-D-Phe-Arg-Trp-Lys]-NH₂,

### H-Gly-Ser-Asn-homoArg-Thr-Ser-Nle-c[Glu-F-Pro-D-Phe-Arg-Trp-Lys]-NH₂,

### H-Gly-Ser-Gln-homoArg-Nle-c[Glu-Hyp-D-Phe-Arg-Trp-Lys]-NH₂,

### H-Gly-Ser-Ser-Tyr-Thr-Nle-c[Glu-Hyp-D-Phe-Arg-Trp-Lys]-NH₂,

### H-Gly-Ser-Asn-Asn-Thr-Nle-c[Glu-Hyp-D-Phe-Arg-Trp-Lys]-NH₂,

### H-Gly-Ser-Ser-homoArg-Thr-Nle-c[Glu-Hyp-D-Phe-Arg-Trp-Lys]-NH₂,

### H-Gly-Ser-Ser-His-Thr-Nle-c[Glu-Hyp-D-Phe-Arg-Trp-Lys]-NH₂,

### H-Gly-Ser-D-Asn-His-Thr-Nle-c[Glu-Hyp-D-Phe-Arg-Trp-Lys]-NH₂,

### H-Gly-Ser-Asn-homoArg-Thr-Nle-c[Glu-Hyp-D-Phe-Arg-Trp-Lys]-NH₂,

### H-Ser-homoArg-Ser-Nle-c[Glu-Met(O2)-D-Phe-Arg-Trp-Lys]-NH₂,

### H-Gln-homoArg-Thr-Nle-c[Glu-Met(O2)-D-Phe-Arg-Trp-Lys]-NH₂,

### H-Ser-homoArg-Thr-Nle-c[Glu-Met(O2)-D-Phe-Arg-Trp-Lys]-NH₂,

### H-Ser-His-Thr-Nle-c[Glu-Met(O2)-D-Phe-Arg-Trp-Lys]-NH₂, and

### H-Ser-homoArg-Thr-Nle-c[Glu-Met(O2)-D-Phe-Arg-Trp-Lys]-NH₂.

Another aspect of the invention relates to a compound of formula II selected from the group consisting of

19-(methylphosphono)nonadecanoic acid,
phosphoric acid 15-carboxypentadecyl pentyl diester,
phosphoric acid 15-carboxypentadecyl octadecyl diester, and
phosphoric acid 15-carboxypentadecyl dodecyl diester.

A further aspect of the invention relates to a compound selected from the group consisting of
(S)-4-[16-{(hydroxy)(octadecyloxy)phosphoryloxy}hexadecanoylamino]-4-(tert-butoxycarbonyl)butyric acid,
(S)-4-[16-{(hydroxy)(dodecyloxy)phosphoryloxy}hexadecanoylamino]-4-(tert-butoxycarbonyl)butyric acid,
(S)-4-[16-{(hydroxy)(pentyloxy)phosphoryloxy}hexadecanoylamino]-4-(tert-butoxycarbonyl)butyric acid, and
(S)-4-[16-{(hydroxy)(methoxy)phosphoryl}nonadecanoylamino]-4-(tert-butoxycarbonyl)butyric acid.

A further aspect of the invention relates to a compound of formula I selected from the group consisting of
N-ε²⁶-((S)-4-[16-{(hydroxy)(octadecyloxy)phosphoryloxy}hexadecanoylamino]-4-carboxybutyryl)[Aib8, Arg34]GLP-1(7-37),
N-ε²⁶-((S)-4-[16-{(hydroxy)(pentyloxy)phosphoryloxy}hexadecanoylamino]-4-carboxybutyryl)[Aib8, Arg34]GLP-1(7-37),
N-ε²⁶-((S)-4-[16-{(hydroxy)(dodecyloxy)phosphoryloxy}hexadecanoylamino]-4-carboxybutyryl)[Aib8, Arg34]GLP-1(7-37),
N-ε²⁶-((S)-4-[16-{(hydroxy)(methoxy)phosphoryl}nonadecanoylamino]-4-carboxybutyryl)[Aib8, Arg34]GLP-1(7-37),
(3-(2-{2-[2-(Hexadecyloxy-hydroxy-phosphoryloxy)-ethoxy]-ethoxy}-ethoxy)-propionyl)-Ser-Nle-c[Glu-Hyp-D-Phe-Arg-Trp-Lys]-NH2,
(3-(2-{2-[2-(Hexadecyloxy-hydroxy-phosphoryloxy)-ethoxy]-ethoxy}-ethoxy)-propionyl)-Arg-Nle-c[Glu-Hyp-D-Phe-Arg-Trp-Lys]-NH2, and
N-Epsilon26-(3-(2-{2-[2-(Hexadecyloxy-hydroxy-phosphoryloxy)-ethoxy]-ethoxy}-ethoxy)-propionyl)-[Aib8, Arg34]GLP-1(7-37).

In another aspect the present invention provides a compound according to formula (I), wherein the molecule is human growth hormone or an analog thereof.

In another aspect the present invention provides a compound according to formula (I), wherein the molecule is factor VII or an analog thereof.

In another aspect the present invention provides a compound according to formula (I), wherein the molecule is parathyroid hormone or an analog thereof.

In another aspect the present invention provides a compound according to formula (I), wherein the molecule is human follicle stimulating hormone or an analog thereof.

In another embodiment the present invention provides a compound according to formula (I), wherein the molecule has a molar weight of less than 100 kDa, less than 50 kDa, or less than 10 kDa.

In other aspects the present invention provides a compound according to formula (I), wherein the molecule is selected from the group consisting of a growth factor such as platelet-derived growth factor (PDGF), Obestatin, transforming growth factor α (TGF-α), transforming growth factor β (TGF-β), epidermal growth factor (EGF), vascular endothelial growth factor (VEGF), a somatomedin such as insulin growth factor I (IGF-I), insulin growth factor II (IFG-II), erythropoietin (EPO), thrombopoietin (TPO) or angiopoietin, interferon, pro-urokinase, urokinase, tissue plasminogen activator (t-PA), plasminogen activator inhibitor 1, plasminogen activator inhibitor 2, von Willebrandt factor, a cytokine, e.g. an interleukin such as interleukin (IL) 1, IL-1Ra, IL-2, IL-4, IL-5, IL-6, IL-9, IL-11, IL-12, IL-13, IL-15, IL-16, IL-17, IL-18, IL-20 or IL-21, a colony stimulating factor (CFS) such as GM-CSF, stem cell factor, a tumor necrosis factor such as TNF-α, lymphotoxin-α, lymphotoxin-β, CD40L, or CD30L, a protease inhibitor e.g. aprotinin, an enzyme such as superoxide dismutase, asparaginase, arginase, arginine deaminase, adenosine deaminase, ribonuclease, catalase, uricase, bilirubin oxidase, trypsin, papain, alkaline phosphatase, β-glucoronidase, purine nucleoside phosphorylase or batroxobin, an opioid, e.g. endorphins, enkephalins or non-natural opioids, a hormone or neuropeptide, e.g. calcitonin, glucagon, gastrins, adrenocorticotropic hormone (ACTH), cholecystokinins, lutenizing hormone, gonadotropin-releasing hormone, chorionic gonadotropin, corticotrophin-releasing factor, vasopressin, oxytocin, antidiuretic hormones, thyroid-stimulating hormone, thyrotropin-releasing hormone, relaxin, prolactin, peptide YY, neuropeptide Y, pancreastic polypeptide, leptin, CART (cocaine and amphetamine regulated transcript), a CART related peptide, perilipin, peptide hormones acting on the melanocortin receptors such as α-MSH or ACTH, melanin-concentrating hormones, natriuretic peptides, adrenomedullin, endothelin, secretin, amylin, vasoactive intestinal peptide (VIP), pituary adenylate cyclase activating polypeptide (PACAP), bombesin, bombesin-like peptides, thymosin, heparin-binding protein, soluble CD4, hypothalmic releasing factor, melanotonins and analogs thereof.

One aspect of the invention provides a method for increasing the plasma half-life of a molecule, comprising covalently linking said molecule to a protractor compound of the general formula II according to the invention.

Another aspect of the invention provides a method for increasing the plasma half-life of a molecule, comprising converting said molecule into a compound of the general formula (I) according to the invention.

Another aspect of the invention provides the use of a compound of the general formula II according to the invention for modifying the pharmacokinetic properties of a therapeutic polypeptide by derivatization of said therapeutic polypeptide with said compound of the general formula II.

Another aspect of the invention provides a pharmaceutical composition comprising a compound of the general formula I according to the invention and a pharmaceutically acceptable excipient.

Another aspect of the invention provides a pharmaceutical composition according to the invention which is suited for parenteral administration.

Another aspect of the invention provides the use of a compound of the general formula I according to the invention for the preparation of a medicament.

Another aspect of the invention provides the use of a compound of the general formula I according to the invention for the preparation of a medicament for the treatment or prevention of hyperglycemia, type 2 diabetes, impaired glucose tolerance, type 1 diabetes, obesity, hypertension, syndrome X, dyslipidemia, cognitive disorders, atheroschlerosis, myocardial infarction, coronary heart disease and other cardiovascular disorders, stroke, inflammatory bowel syndrome, dyspepsia and gastric ulcers.

Another aspect of the invention provides the use of a compound of the general formula I according to the invention for the preparation of a medicament for delaying or preventing disease progression in type 2 diabetes.

Another aspect of the invention provides the use of a compound of the general formula I according to the invention for the preparation of a medicament for decreasing food intake, decreasing β-cell apoptosis, increasing β-cell function and β-cell mass, and/or for restoring glucose sensitivity to β-cells.

Another aspect of the invention provides the use of a compound of the general formula I according to the invention for the preparation of a medicament for the treatment of small bowel syndrome, inflammatory bowel syndrome or Crohns disease.

Another aspect of the invention provides the use of a compound of the general formula I according to the invention for the preparation of a medicament for the treatment or prevention of hyperglycemia, type 1 diabetes, type 2 diabetes or β-cell deficiency.

The therapeutic polypeptides can be produced by classical peptide synthesis, e.g. solid phase peptide synthesis using t-Boc or Fmoc chemistry or other well established techniques, see e.g. Green and Wuts, “Protecting Groups in Organic Synthesis”, John Wiley & Sons, 1999.

The therapeutic polypeptides can also be produced by a method which comprises culturing a host cell containing a DNA sequence encoding the polypeptide and capable of expressing the polypeptide in a suitable nutrient medium under conditions permitting the expression of the peptide, after which the resulting peptide is recovered from the culture.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The peptide produced by the cells may then be recovered from the culture medium by conventional procedures including separating the host cells from the medium by centrifugation or filtration. For extracellular products the proteinaceous components of the supernatant are isolated by filtration, column chromatography or precipitation, e.g. microfiltration, ultrafiltration, isoelectric precipitation, purification by a variety of chromatographic procedures, e.g. ion exchange chromatography, hydrophobic interaction chromatography, gel filtration chromatography, affinity chromatography, or the like, dependent on the type of polypeptide in question. For intracellular or periplasmic products the cells isolated from the culture medium are disintegrated or permeabilised and extracted to recover the product polypeptide or precursor thereof.

The DNA sequence encoding the therapeutic polypeptide may suitably be of genomic or cDNA origin, for instance obtained by preparing a genomic or cDNA library and screening for DNA sequences coding for all or part of the peptide by hybridisation using synthetic oligonucleotide probes in accordance with standard techniques (see, for example, Sambrook, J, Fritsch, E F and Maniatis, T, Molecular Cloning: A Laboratory Manual, Cold Spring Harbor Laboratory Press, New York, 1989). The DNA sequence encoding the polypeptide may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. The DNA sequence may also be prepared by polymerase chain reaction using specific primers, for instance as described in U.S. Pat. No. 4,683,202 or Saiki et al., Science 239 (1988), 487-491.

The DNA sequence may be inserted into any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the polypeptide is operably linked to additional segments required for transcription of the DNA, such as a promoter. The promoter may be any DNA sequence which shows transcriptional activity in the host cell of choice and may be derived from genes encoding proteins either homologous or heterologous to the host cell. Examples of suitable promoters for directing the transcription of the DNA encoding the peptide of the invention in a variety of host cells are well known in the art, cf. for instance Sambrook et al., supra.

The DNA sequence encoding the polypeptide may also, if necessary, be operably connected to a suitable terminator, polyadenylation signals, transcriptional enhancer sequences, and translational enhancer sequences. The recombinant vector of the invention may further comprise a DNA sequence enabling the vector to replicate in the host cell in question.

The vector may also comprise a selectable marker, e.g. a gene the product of which complements a defect in the host cell or one which confers resistance to a drug, e.g. ampicillin, kanamycin, tetracyclin, chloramphenicol, neomycin, hygromycin or methotrexate. For large scale manufacture the selectable marker preferably is not antibiotic resistance, e.g. antibiotic resistance genes in the vector are preferably excised when the vector is used for large scale manufacture. Methods for eliminating antibiotic resistance genes from vectors are known in the art, see e.g. U.S. Pat. No. 6,358,705 which is incorporated herein by reference.

To direct a parent peptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the peptide in the correct reading frame. Secretory signal sequences are commonly positioned 5′ to the DNA sequence encoding the peptide. The secretory signal sequence may be that normally associated with the peptide or may be from a gene encoding another secreted protein.

The procedures used to ligate the DNA sequences coding for the present peptide, the promoter and optionally the terminator and/or secretory signal sequence, respectively, and to insert them into suitable vectors containing the information necessary for replication, are well known to persons skilled in the art (cf., for instance, Sambrook et al., supra).

The host cell into which the DNA sequence or the recombinant vector is introduced may be any cell which is capable of producing the present peptide and includes bacteria, yeast, fungi and higher eukaryotic cells. Examples of suitable host cells well known and used in the art are without limitation, E. coli, Saccharomyces cerevisiae, or mammalian BHK or CHO cell lines.

For connecting the protracting tag to a molecule of interest, such as a therapeutically effective polypeptide, various different strategies may be envisioned.

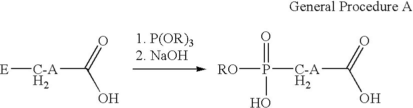

A carboxylic acid of the general formula E-CH₂-A-CO₂H, in which E represents a leaving group for nucleophilic displacement, such as halogen, lower alkyl sulfonate (e.g. MeSO₂—O—), aryl sulfonate, dialkylsulfonium, diarylsulfonium, hydroxonium, or the like, and A is as defined above, is heated with an excess of a trialkyl phosphite P(OR)₃. When all the starting acid is consumed, the excess phosphite is evaporated off, and the residue is mixed with an aqueous solution of an alkali metal hydroxide, such as sodium, potassium, or lithium hydroxide, or mixtures thereof, optionally in the presence of a cosolvent, such as ethanol, ethylene glycol, or the like, and stirred while heating until all the intermediate dialkyl phosphonate is consumed. The product can be isolated by acidification with a strong mineral acid, such as hydrochloric acid, filtration, and recrystallization of the solid from a suitable solvent, such as acetonitrile.

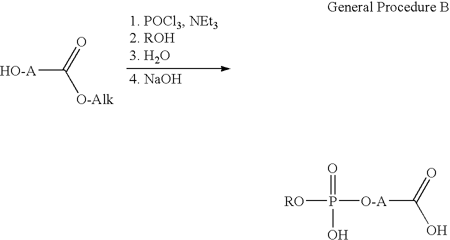

To a solution of an omega-hydroxyalkanoic acid alkyl ester of the general formula HO-A-CO₂Alk, in which Alk is lower alkyl and A is as defined above, in a dry, non-nucleophilic solvent, such as dichloromethane, chlorobenzene, 1,2-dichloroethane, chloroform, carbon tetrachloride, carbon disulfide, or the like, at 0° C., are added 1.2 equivalents of a tertiary amine, such as triethylamine, and then POCl₃ (1.1 equivalents). The resulting mixture is stirred at 0° C. for 1-5 h, and a solution of an alcohol ROH (1.2 equivalents) in a dry, non-nucleophilic solvent, such as dichloromethane, is added. After stirring overnight at room temperature, pyridine (5 equivalents) is added, followed one hour later by the addition of an excess of water. The resulting mixture is stirred at room temperature for 3 h, acidified by addition of a strong mineral acid, such as hydrochloric acid, the organic solvents evaporated off under reduced pressure, and the ester isolated by filtration. The crude ester may be purified by recrystallization from a suitable solvent, such as acetonitrile.

This intermediate is mixed with an aqueous solution of an alkali metal hydroxide, such as sodium, potassium, or lithium hydroxide, or mixtures thereof, optionally in the presence of a cosolvent, such as methanol, ethanol, ethylene glycol, or the like, and stirred while heating until all the intermediate ester is consumed. Acidification with a strong acid, followed by filtration and recrystallization from a suitable solvent yields the omega-carboxyalkyl(alkyl)phosphates.

### General Procedure C for the Synthesis of Protected Phosphordiesters Using 2-Cyanoethyl N,N-diisopropyl-chlorophosphoramidite Chemistry.

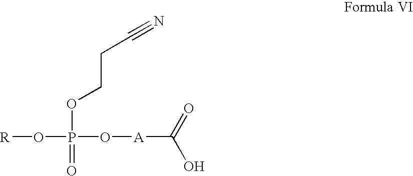

1 equivalent of the alcohol R—OH and 1.1 equivalent of triethylamine are dissolved in THF (˜0.5 mmol/mL). 1 equivalent of 2-Cyanoethyl N,N-diisopropyl-chlorophosphoramidite is added and the mixture is stirred under nitrogen atmosphere for at least 1 hour but not longer than 16 hours. 1 equivalent of HO-A-C(O)OX, X represents an acid labile protecting group like tert.butyl, and 1.1 equivalent of tetrazol as a solution of tetrazol in acetonitril are added. The mixture is stirred again for 2-4 hours.

The phosphor is then oxidized using tert.butyl hydroperoxide in nonane (5.5M, 5 equivalents). A iodine solution in THF/2.6-lutidin/water 7/2/1 can also be used. The mixture is stirred for an additional 1-4 hours and then poured into ethylacetate and washed with 2% sodiumsulfite in water. The ethylacetate phase is dried over sodiumsulfate or magnesiumsulfate and the solvent is removed in vacuum. The product can be purified by chromatography on silica using an appropriate solvent mixture like ethylacetate/hexane.

The crude or purified product is dissolved in TFA is cleave the acid labile protecting group.

The final product can be purified by chromatography on silica using an appropriate solvent mixture like ethylacetate and 1% acetic acid.

General Procedure D for the Coupling of the Protected Phosphordiester onto a Peptide on a Solid Phase

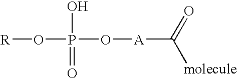

The protected phosphordiester of formula molecule VI can be coupled onto a free amine by activating the carboxylic acid under standard condition using 1 equivalent of any carbodiimide (preferred N,N′-diisopropylcarbodiimide) and 1 equivalent 1-hydroxybenzotriazol in NMP. If X is coupled to a peptide on solid phase, 2-4 equivalents in relation to the peptide can be used. After a successful coupling, the resin with the peptide is washed with NMP and the phosphor protecting group is removed by treating the resin with a 20% piperidine in NMP solution for 30 min. The resin is washed again with NMP and dichloromethane. The peptide is then cleaved and purified under standard conditions as described elsewhere and known to the ones skilled in the art.

Another object of the present invention is to provide a pharmaceutical formulation comprising a compound according to the invention which is present in a concentration from about 0.1 mg/ml to about 25 mg/ml, and wherein said formulation has a pH from 2.0 to 10.0. The formulation may further comprise a buffer system, preservative(s), tonicity agent(s), chelating agent(s), stabilizers and surfactants. In one aspect of the invention the pharmaceutical formulation is an aqueous formulation, i.e. formulation comprising water. Such formulation is typically a solution or a suspension. In a further aspect of the invention the pharmaceutical formulation is an aqueous solution. The term “aqueous formulation” is defined as a formulation comprising at least 50%/w/w water. Likewise, the term “aqueous solution” is defined as a solution comprising at least 50%/w/w water, and the term “aqueous suspension” is defined as a suspension comprising at least 50%/w/w water.

In another aspect the pharmaceutical formulation is a freeze-dried formulation, whereto the physician or the patient adds solvents and/or diluents prior to use.

In another aspect the pharmaceutical formulation is a dried formulation (e.g. freeze-dried or spray-dried) ready for use without any prior dissolution.

In a further aspect the invention relates to a pharmaceutical formulation comprising an aqueous solution of a compound according to the invention with formula I, and a buffer, wherein said compound according to the invention with formula I is present in a concentration from 0.1 mg/ml or above, and wherein said formulation has a pH from about 2.0 to about 10.0.

In a another aspect of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In a further aspect of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further aspect of the invention the formulation further comprises a pharmaceutically acceptable preservative. In a further aspect of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further aspect of the invention the preservative is present in a concentration from 0.1 mg/ml to 20 mg/ml. In a further aspect of the invention the preservative is present in a concentration from 0.1 mg/ml to 5 mg/ml. In a further aspect of the invention the preservative is present in a concentration from 5 mg/ml to 10 mg/ml. In a further aspect of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19th edition, 1995.

In a further aspect of the invention the formulation further comprises an isotonic agent. In a further aspect of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one aspect the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one aspect the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one aspect, the sugar or sugar alcohol concentration is between about 1 mg/ml and about 150 mg/ml. In a further aspect of the invention the isotonic agent is present in a concentration from 1 mg/ml to 50 mg/ml. In a further aspect of the invention the isotonic agent is present in a concentration from 1 mg/ml to 7 mg/ml. In a further aspect of the invention the isotonic agent is present in a concentration from 8 mg/ml to 24 mg/ml. In a further aspect of the invention the isotonic agent is present in a concentration from 25 mg/ml to 50 mg/ml. Each one of these specific isotonic agents constitutes an alternative aspects of the invention. The use of an isotonic agent in pharmaceutical compositions is well-known to the skilled person. For convenience reference is made to Remington: The Science and Practice of Pharmacy, 19th edition, 1995.

In a further aspect of the invention the formulation further comprises a chelating agent. In a further aspect of the invention the chelating agent is selected from salts of ethylenediaminetetraacetic acid (EDTA), citric acid, and aspartic acid, and mixtures thereof. In a further aspect of the invention the chelating agent is present in a concentration from 0.1 mg/ml to 5 mg/ml. In a further aspect of the invention the chelating agent is present in a concentration from 0.1 mg/ml to 2 mg/ml. In a further aspect of the invention the chelating agent is present in a concentration from 2 mg/ml to 5 mg/ml. Each one of these specific chelating agents constitutes an alternative aspect of the invention. The use of a chelating agent in pharmaceutical compositions is well-known to the skilled person. For convenience reference is made to Remington: The Science and Practice of Pharmacy, 19th edition, 1995.

In a further aspect of the invention the formulation further comprises a stabilizer. The use of a stabilizer in pharmaceutical compositions is well-known to the skilled person. For convenience reference is made to Remington: The Science and Practice of Pharmacy, 19th edition, 1995.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one aspect, amino acids to use in preparing the compositions of the invention are those carrying a charged side chain, such as arginine, lysine, aspartic acid, and glutamic acid. Any stereoisomer (i.e., L, D, or a mixture thereof) of a particular amino acid (e.g. methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further aspect of the invention the amino acids or amino acid analogues are used in a concentration, which is sufficient to prevent or delay aggregation of the protein.

In a further aspect of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L or D) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further aspect of the invention the formulation further comprises a stabilizer selected from the group of high molecular weight polymers or low molecular compounds. In a further aspect of the invention the stabilizer is selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative aspect of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further aspect of the invention the formulation further comprises a surfactant. In a further aspect of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)—derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C6-C12 (eg. oleic acid and caprylic acid), acylcarnitines and derivatives, Nα-acylated derivatives of lysine, arginine or histidine, or side-chain acylated derivatives of lysine or arginine, Nα-acylated derivatives of dipeptides comprising any combination of lysine, arginine or histidine and a neutral or acidic amino acid, Nα-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative aspect of the invention.

The use of a surfactant in pharmaceutical compositions is well-known to the skilled person. For convenience reference is made to Remington: The Science and Practice of Pharmacy, 19th edition, 1995.

In a further aspect of the invention the formulation further comprises protease inhibitors such as EDTA (ethylenediamine tetraacetic acid) and benzamidineHCl, but other commercially available protease inhibitors may also be used. The use of a protease inhibitor is particular useful in pharmaceutical compositions comprising zymogens of proteases in order to inhibit autocatalysis.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a compound according to the invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the a compound according to the invention, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of a compound according to the invention, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the compound according to the invention with formula I in the form of a nasal or pulmonal spray. As a still further option, the pharmaceutical compositions containing the compound according to the invention with formula I can also be adapted to transdermal administration, e.g. by needle-free injection or from a patch, optionally an iontophoretic patch, or transmucosal, e.g. buccal, administration.

The compound according to the invention can be administered via the pulmonary route in a vehicle, as a solution, suspension or dry powder using any of known types of devices suitable for pulmonary drug delivery. Examples of these comprise of, but are not limited to, the three general types of aerosol-generating for pulmonary drug delivery, and may include jet or ultrasonic nebulizers, metered-dose inhalers, or dry powder inhalers (Cf. Yu J, Chien Y W. Pulmonary drug delivery: Physiologic and mechanistic aspects. Crit Rev Ther Drug Carr Sys 14(4) (1997) 395-453).

Based on standardised testing methodology, the aerodynamic diameter (da) of a particle is defined as the geometric equivalent diameter of a reference standard spherical particle of unit density (1 g/cm3). In the simplest case, for spherical particles, da is related to a reference diameter (d) as a function of the square root of the density ratio as described by:

$${d}_{a} = \sqrt{\frac{ρ}{{ρ}_{a}}} d$$

Modifications to this relationship occur for non-spherical particles (cf. Edwards D A, Ben-Jebria A, Langer R. Recent advances in pulmonary drug delivery using large, porous inhaled particles. J Appl Physiol 84(2) (1998) 379-385). The terms “MMAD” and “MMEAD” are well-described and known to the art (cf. Edwards D A, Ben-Jebria A, Langer R and represents a measure of the median value of an aerodynamic particle size distribution. Recent advances in pulmonary drug delivery using large, porous inhaled particles. J Appl Physiol 84(2) (1998) 379-385). Mass median aerodynamic diameter (MMAD) and mass median effective aerodynamic diameter (MMEAD) are used inter-changeably, are statistical parameters, and empirically describe the size of aerosol particles in relation to their potential to deposit in the lungs, independent of actual shape, size, or density (cf. Edwards D A, Ben-Jebria A, Langer R. Recent advances in pulmonary drug delivery using large, porous inhaled particles. J Appl Physiol 84(2) (1998) 379-385). MMAD is normally calculated from the measurement made with impactors, an instrument that measures the particle inertial behaviour in air.

In a further aspect of the invention, the formulation could be aerosolized by any known aerosolisation technology, such as nebulisation, to achieve a MMAD of aerosol particles less than 10 μm, more preferably between 1-5 μm, and most preferably between 1-3 μm. The preferred particle size is based on the most effective size for delivery of drug to the deep lung, where protein is optimally absorbed (cf. Edwards D A, Ben-Jebria A, Langer A, Recent advances in pulmonary drug delivery using large, porous inhaled particles. J Appl Physiol 84(2) (1998) 379-385).

Deep lung deposition of the pulmonal formulations comprising the compound according to the invention may optional be further optimized by using modifications of the inhalation techniques, for example, but not limited to: slow inhalation flow (eg. 30 L/min), breath holding and timing of actuation.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one aspect of the invention the pharmaceutical formulation comprising the compound according to the invention with formula I is stable for more than 6 weeks of usage and for more than 3 years of storage.

In another aspect of the invention the pharmaceutical formulation comprising the compound according to the invention with formula I is stable for more than 4 weeks of usage and for more than 3 years of storage.

In a further aspect of the invention the pharmaceutical formulation comprising the compound according to the invention with formula I is stable for more than 4 weeks of usage and for more than two years of storage.

In an even further aspect of the invention the pharmaceutical formulation comprising the compound according to the invention with formula I is stable for more than 2 weeks of usage and for more than two years of storage.

In another aspect the present invention relates to the use of a compound according to the invention for the preparation of a medicament.

In one aspect of the invention a compound according to the invention wherein the therapeutic agent is a GLP-1 peptide is used for the preparation of a medicament for the treatment or prevention of hyperglycemia, type 2 diabetes, impaired glucose tolerance, type 1 diabetes, obesity, hypertension, syndrome X, dyslipidemia, cognitive disorders, atheroschlerosis, myocardial infarction, coronary heart disease and other cardiovascular disorders, stroke, inflammatory bowel syndrome, dyspepsia and gastric ulcers.

In another aspect of the invention a compound according to the invention wherein the therapeutic agent is a GLP-1 peptide is used for the preparation of a medicament for delaying or preventing disease progression in type 2 diabetes.

In another aspect of the invention a compound according to the invention wherein the therapeutic agent is a GLP-1 peptide is used for the preparation of a medicament for decreasing food intake, decreasing β-cell apoptosis, increasing β-cell function and β-cell mass, and/or for restoring glucose sensitivity to β-cells.

The treatment with a compound according to the present invention may also be combined with a second or more pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. Examples of these pharmacologically active substances are: Insulin, Obestatin, GLP-1 agonists, sulphonylureas, biguanides, meglitinides, glucosidase inhibitors, glucagon antagonists, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents as HMG CoA inhibitors (statins), compounds lowering food intake, RXR agonists and agents acting on the ATP-dependent potassium channel of the β-cells; Cholestyramine, colestipol, clofibrate, gemfibrozil, lovastatin, pravastatin, simvastatin, probucol, dextrothyroxine, neteglinide, repaglinide; β-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin; CART (cocaine amphetamine regulated transcript) agonists, NPY (neuropeptide Y) antagonists, MC4 (melanocortin 4) agonists or antagonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists; histamine H3 antagonists.

It should be understood that any suitable combination of the compounds according to the invention with one or more of the above-mentioned compounds and optionally one or more further pharmacologically active substances are considered to be within the scope of the present invention.

The present invention is further illustrated by the following examples which, however, are not to be construed as limiting the scope of protection. The features disclosed in the foregoing description and in the following examples may, both separately and in any combination thereof, be material for realizing the invention in diverse forms thereof.

### EXAMPLES

In the examples the following terms are intended to have the following, general meanings:

Boc: tert-butyloxycarbonyl
DBU: 1,8-diazabicyclo[5.4.0]undec-7-ene
DCM: dichloromethane, methylenechloride
Dde: 1-(4,4-dimethyl-2,6-dioxocyclohexylidene)ethyl
DIC: diisopropylcarbodiimide
DIPEA diisopropylethylamine

### DMA: N,N-dimethylacetamide

DMF: N,N-dimethyl formamide
DMSO: dimethyl sulfoxide
DMAP: 4-dimethylaminopyridine
DMPU: 1,3-dimethyltetrahydropyrimidin-2-one
EDC or EDAC: N-ethyl-N′-(3-dimethylaminopropyl)carbodiimide hydrochloride
Fmoc: 9-fluorenylmethyloxycarbonyl
HBTU: 2-(1H-Benzotriazol-1-yl-)-1,1,3,3 tetramethyluronium hexafluorophosphate
HOAt: 3-hydroxy-3H-[1,2,3]triazolo[4,5-b]pyridine, 4-aza-3-hydroxybenzotriazole
HOBt: N-hydroxybenzotriazole, 1-hydroxybenzotriazole

### HONSU: N-hydroxysuccinimide

### NMP: N-methylpyrrolidone

HPLC: high pressure liquid chromatography
pbf: 2,2,4,6,7-pentamethyl-2,3-dihydrobenzo[b]furan-5-sulfonyl
Pmc 2,2,5,7,8-pentamethylchroman-6-sulfonyl
r.t. room temperature
Su: succinimidyl
TIS: triisopropylsilane
Tmob: 2,4,6-trimethoxybenzyl
Trt: trityl, triphenylmethyl
Ts: toluenesulfonyl
TSTU: O-(1-succinimidyl)-N,N,N′,N′-tetramethyluronium hexafluorophosphate
CH₃CN or MeCN: acetonitrile
OtBu: tert butyl ester
tBu: tert butyl
Trt: triphenylmethyl
Dde: 1-(4,4-Dimethyl-2,6-dioxocyclohexylidene)ethyl
TFA: trifluoroacetic acid
Et₂O: diethylether

### Example 1

### 19-(Methylphosphono)nonadecanoic acid

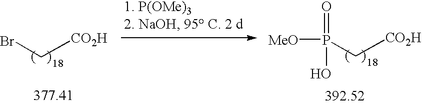

19-Bromononadecanoic acid (1.0 g, 2.65 mmol) was mixed with trimethylphosphite (15 ml) and heated to reflux (117° C.) for 66 h. The mixture was concentrated under reduced pressure, and the residue was mixed with water (10 ml), LiOH-hydrate (1.90 g), NaOH (2.0 g), and EtOH (10 ml). The mixture was stirred at 95° C. (oil-bath temperature) for 2 d. More water (10 ml) and EtOH (10 ml) were added, and heating was continued for 24 h.

The mixture was diluted with water (150 ml) and conc. HCl (10 ml), stirred for 1 h, and the product was filtered off and washed with water. Recrystallization from boiling MeCN (20 ml) yielded 0.75 g (72%) of the title compound as a solid.

¹H NMR (DMSO-d₆): δ 1.23 (s, 28H), 1.25-1.62 (m, 6H), 2.18 (t, J=7 Hz, 2H), 3.51 (d, J=10 Hz, 3H).

### Example 2

### Phosphoric acid 15-carboxypentadecyl pentyl diester

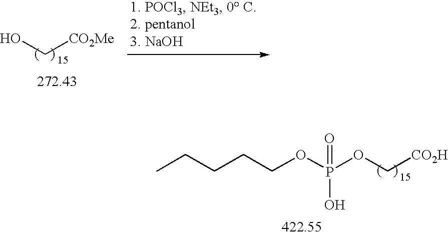

16-Hydroxyhexadecanoic acid methyl ester (0.57 g, 2 mmol) was dissolved in warm toluene (50 ml) and concentrated under reduced pressure to remove water and residual MeOH. The residue was dissolved in DCM (25 ml), and at 0° C. under nitrogen were added NEt₃ (0.335 ml, 2.42 mmol) and then POCl₃ (0.205 ml, 2.21 mmol) in one portion. After stirring at 0° C. for 1 h pentanol (0.26 ml, 2.39 mmol) was added. Stirring was continued while allowing the ice-bath to melt. After 22 h pyridine (0.9 ml, 11 mmol) was added. After 1 h water (30 ml) was added and stirring was continued for 3 h. 1N HCl (40 ml) was added, and the DCM was evaporated off under reduced pressure. The residual suspension was kept at room temperature overnight, filtered, and the solid was washed with water. Recrystallization from hot MeCN (20 ml) yielded 0.24 g of a solid. Analysis by ¹H NMR suggested this product to be a mixture of the expected methyl ester and the corresponding acid.

The saponification was completed by adding to this product MeOH (10 ml) and a solution of NaOH (1.1 g, 27.5 mmol) in water (2.0 ml). The mixture was stirred at 65° C. for 19 h, diluted with water (50 ml) and acidified with conc. HCl. After stirring for 3 h the mixture was filtered, the solid was washed with water and purified by recrystallization from hot MeCN (20 ml) to yield 0.12 g (14%) of the title compound as a solid.

¹H NMR (DMSO-d₆): δ 0.88 (m, 3H), 1.24 (m, 28H), 1.42-1.60 (m, 4H), 2.18 (t, J=7 Hz, 2H), 3.83 (m, 4H).

LCMS: MH⁺: 423 (5.6 min).

### Example 3

### Phosphoric acid 15-carboxypentadecyl octadecyl diester

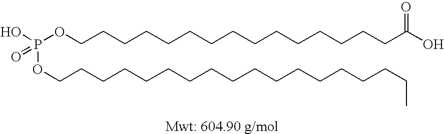

This compound was prepared from 16-hydroxyhexadecanoic acid methyl ester and octadecanol using the same procedure as in the example 2.

¹H NMR (CDCl₃): δ 0.88 (m, 3H), 1.29 (m, 54H), 1.52-1.75 (m, 4H), 2.38 (m, 2H), 4.02 (m, 4H).

### Example 4

### Phosphoric acid 15-carboxypentadecyl dodecyl diester

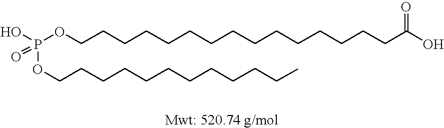

This compound was prepared from 16-hydroxyhexadecanoic acid methyl ester and dodecanol using the same procedure as in the previous example 3.

¹H NMR (DMSO-d₆): δ 0.83 (m, 3H), 1.23 (m, 42H), 1.42-1.60 (m, 4H), 2.18 (t, J=7 Hz, 2H), 3.83 (m, 4H).

LCMS: MH⁺: 521 (7.8 min).

### Example 5

### (S)-4-[16-{(hydroxy)(octadecyloxy)phosphoryloxy}hexadecanoylamino]-4-(tert-butoxycarbonyl)butyric acid

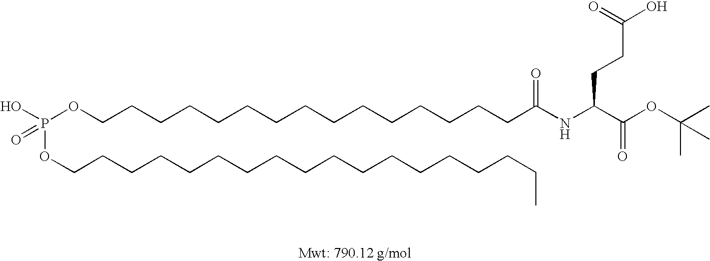

Phosphoric acid 15-carboxypentadecyl octadecyl diester (0.13 g, 215 μmol) and HOBt hydrate (30 mg, 196 μmol) were suspended in DMF (3.0 ml), and DIPEA (0.15 ml, 847 μmol) and DIC (0.034 ml, 217 μmol) were added. The mixture was added to chlorotrityl-polystyrene-esterified glutamic acid tert-butyl ester (166 mg), and shaken at room temperature for 16 h. The resin was filtered off, and washed extensively with DMF and DCM.

The resin was suspended in DCM with 10% TFA, shaken for 15 min, filtered, and the filtrate was concentrated under reduced pressure. The residue was resuspended in warm MeCN, allowed to cool to room temperature, and the product was filtered off and dried under reduced pressure. 15 mg of the title compound was obtained.

¹H NMR (CDCl₃): δ 0.88 (m, 3H), 1.25 (br s, 58H), 1.47 (s, 9H), 1.62 (m, 4H), 2.24 (m, 2H), 2.38 (m, 1H), 3.94 (m, 4H), 6.35 (br d, J=7 Hz, 1H).

### Example 6

### (S)-4-[16-{(hydroxy)(dodecyloxy)phosphoryloxy}hexadecanoylamino]-4-(tert-butoxycarbonyl)butyric acid

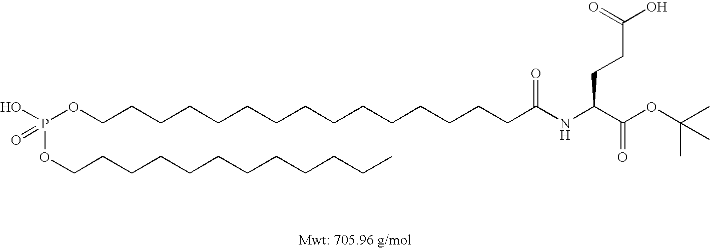

Phosphoric acid 15-carboxypentadecyl dodecyl diester (70 mg, 134 μmol) and HOBt hydrate (22 mg, 144 μmol) were suspended in DMF (1.0 ml), and DIPEA (0.111 ml, 627 μmol) and DIC (0.025 ml, 160 μmol) were added. After 0.5 h the mixture was added to chlorotrityl-polystyrene-esterified glutamic acid tert-butyl ester (120 mg), and shaken at room temperature over night. The resin was filtered off, and washed extensively with DMF and DCM.

The resin was suspended in DCM with 10% TFA, shaken for 20 min, filtered, and the filtrate was concentrated under reduced pressure. 35 mg of the title compound was obtained.

LCMS: MH⁺: 706.

### Example 7

### (S)-4-[16-{(hydroxy)(pentyloxy)phosphoryloxy}hexadecanoylamino]-4-(tert-butoxycarbonyl)butyric acid

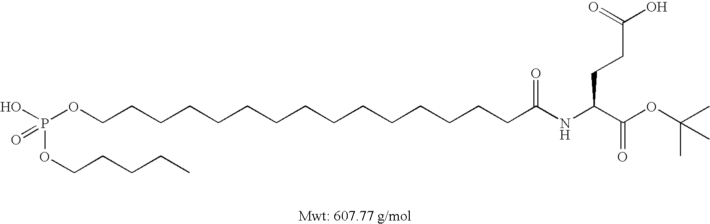

Phosphoric acid 15-carboxypentadecyl dodecyl diester (70 mg, 166 μmol) and HOBt hydrate (22 mg, 144 μmol) were suspended in DMF (1.0 ml), and DIPEA (0.111 ml, 627 μmol) and DIC (0.025 ml, 160 μmol) were added. After 0.5 h the mixture was added to chlorotrityl-polystyrene-esterified glutamic acid tert-butyl ester (120 mg), and shaken at room temperature over night. The resin was filtered off, and washed extensively with DMF and DCM.

The resin was suspended in DCM with 10% TFA, shaken for 20 min, filtered, and the filtrate was concentrated under reduced pressure. 50 mg of the title compound was obtained.

LCMS: MH⁺: 608.

### Example 8

### (S)-4-[16-{(hydroxy)(methoxy)phosphoryl}nonadecanoylamino]-4-(tert-butoxycarbonyl)butyric acid

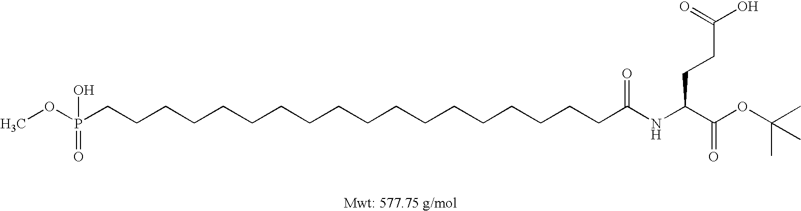

19-(Methylphosphono)nonadecanoic acid (65 mg, 154 μmol) and HOBt hydrate (22 mg, 144 μmol) were suspended in a mixture of DMF (1.0 ml) and DCM (1.0 ml), and DIPEA (0.111 ml, 627 μmol) and DIC (0.025 ml, 160 μmol) were added. After 0.5 h the mixture was added to chlorotrityl-polystyrene-esterified glutamic acid tert-butyl ester (120 mg), and shaken at room temperature over night. The resin was filtered off, and washed extensively with DMF and DCM.

The resin was suspended in DCM with 10% TFA, shaken for 20 min, filtered, and the filtrate was concentrated under reduced pressure. 30 mg of the title compound was obtained.

LCMS: MH⁺: 578.

### Example 9

### N-ε²⁶-((S)-4-[16-{(hydroxy)(octadecyloxy)phosphoryloxy}hexadecanoylamino]-4-carboxybutyryl)[Aib8, Arg34]GLP-1(7-37) (Mwt: 4113.8 g/mol)

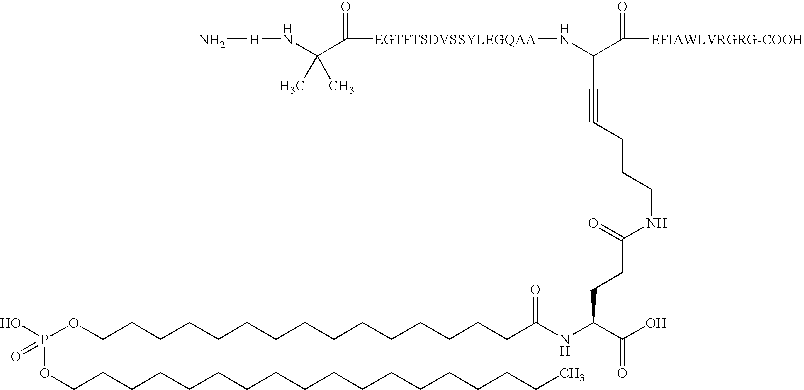

[Aib8, Arg34]GLP-1(7-37) was prepared by standard Fmoc solid phase synthesis, using the following three, backbone-protected dipeptides: Fmoc-Arg(pbf)-(tmob)-Gly-OH, Fmoc-Glu(OtBu)-(tmob)-Gly-OH, and Fmoc-Val-Ser(ψ^{Me,Me}pro)-OH.

(S)-4-[16-{(Hydroxy)(octadecyloxy)phosphoryloxy}hexadecanoylamino]-4-(tert-butoxycarbonyl)butyric acid was dissolved in NMP (0.3 ml), and DIPEA (19 μl, 107 μmol) and TSTU (7 mg, 23 μmol) were added. After stirring for 0.5 h, the mixture was added dropwise to a solution of [Aib8, Arg34]GLP-1(7-37) (85 mg; approx. 25 μmol) and DIPEA (0.1 ml, 563 μmol) in water (2.0 ml). When the reaction was over (HPLC), the mixture was concentrated, and the residue treated for 0.5 h with TFA containing 5% water. The mixture was concentrated and the residue coevaporated with MeCN. Purification by HPLC yielded 11 mg (approx 15%) of the title compound.

LCMS: MH₃³⁺: 1372. 100% pure by HPLC.

### Example 10

### N-ε²⁶-((S)-4-[16-{(hydroxy)(pentyloxy)phosphoryloxy}hexadecanoylamino]-4-carboxybutyryl)[Aib8, Arg34]GLP-1(7-37) (Mwt: 3931.4 g/mol)

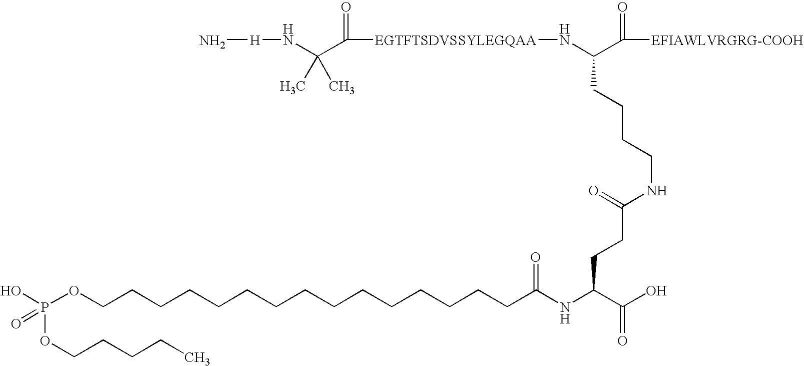

This product was prepared as the previous example. From [Aib8, Arg34]GLP-1(7-37) (80 mg; approx. 24 μmol) was obtained 5.1 mg (approx 6%) of the title compound.

LCMS: MH₃³⁺: 1311. 930% pure by HPLC.

### Example 11

### N-ε²⁶-((S)-4-[16-{(hydroxy)(dodecyloxy)phosphoryloxy}hexadecanoylamino]-4-carboxybutyryl)[Aib8, Arg34]GLP-1(7-37) (Mwt: 4029.6 g/mol)

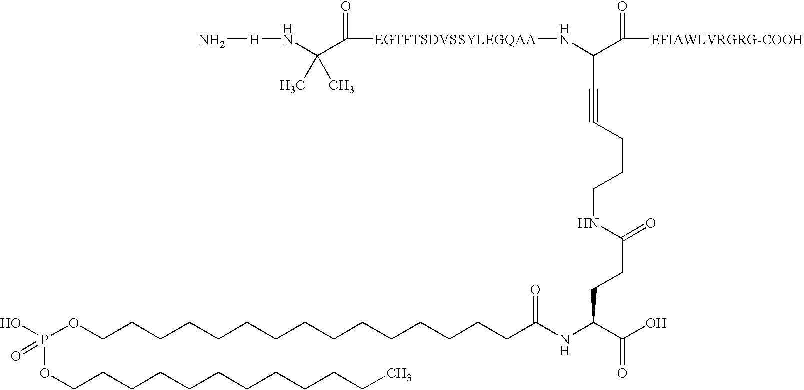

This product was prepared as the previous example. From [Aib8, Arg34]GLP-1(7-37) (80 mg; approx. 24 μmol) was obtained 12.7 mg (approx 15%) of the title compound.

LCMS: MH₃³⁺: 1344. 970% pure by HPLC.

### Example 12

### N-ε²⁶-((S)-4-[16-{(hydroxy)(methoxy)phosphoryl}nonadecanoylamino]-4-carboxybutyryl)[Aib8, Arg34]GLP-1(7-37) (Mwt: 3901.4 g/mol)

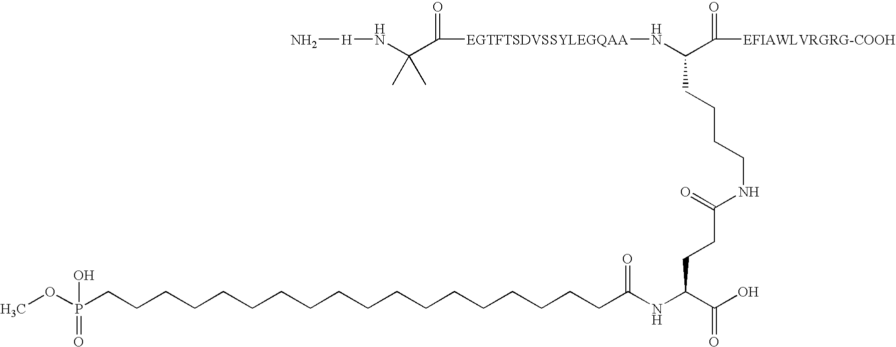

This product was prepared as the previous example. From [Aib8, Arg34]GLP-1(7-37) (80 mg; approx. 24 μmol) was obtained 10.9 mg (approx 13%) of the title compound.

LCMS: MH₃³⁺: 1344. >950% pure by HPLC.

### Example 13

### Synthesis of 3-[2-(2-{2-[(2-Cyano-ethoxy)-hexadecyloxy-phosphoryloxy]-ethoxy}-ethoxy)-ethoxy]-propionic acid (A)

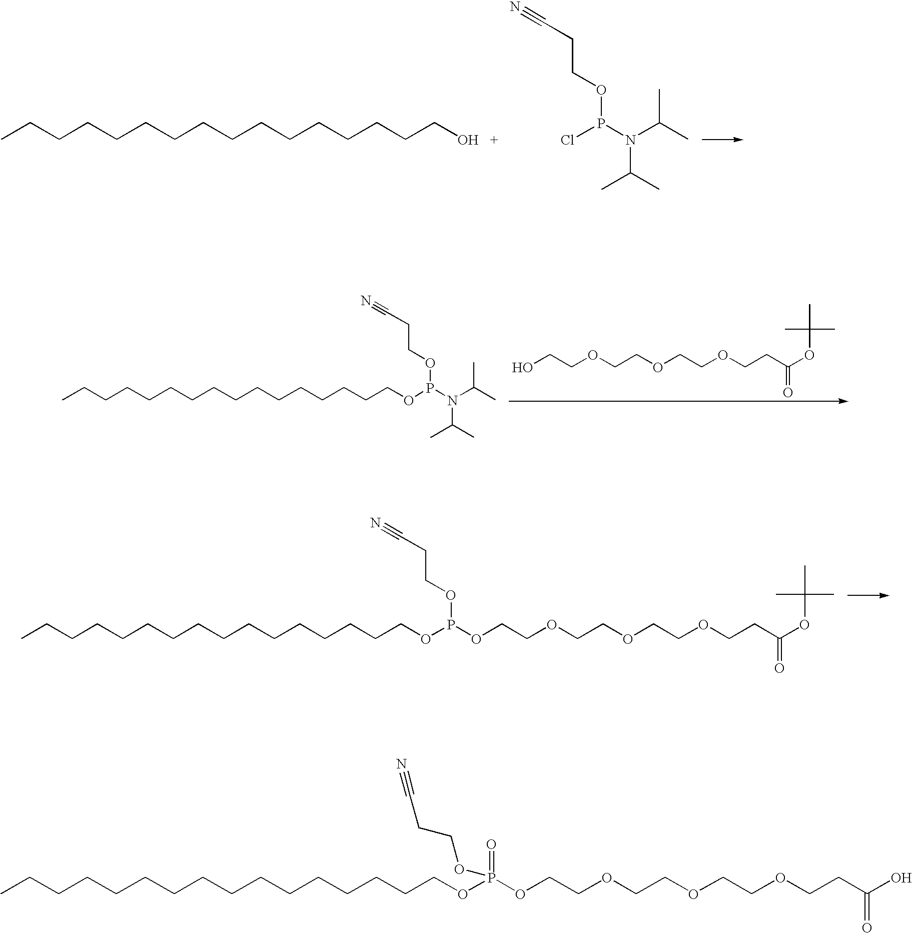

5.1 g of hexadecanol was dissolved in 40 mL of tetrahydrofuran (THF) and added 4.5 mL triethylamine (TEA). The solution was cooled with ice and 5 g of 2-Cyanoethyl N,N-diisopropyl-chlorophosphoramidite was added. The mixture was stirred under nitrogen for 1 h. Then the white precipitate (triethylammonium chloride) was filtered off and washed once with THF.

60 mL of 0.25M tetrazol in acetonitrile and 5 g of tert-Butyl 12-hydroxy-4,7,10-trioxadodecanoate were added and the reaction was stirred at room temperature for 5 h. 7.8 g of iodine was dissolved in 100 mL THF/2.6-lutidin/water 7/2/1 and added to the reaction, which was stirred then over night.

Natriumsulfite solution was added to the reaction mixture until the colour of iodine disappeared and the product was distracted with ethylacetate. The org. phase was washed with sat. sodiumcarbonate and sat. sodiumchloride solution.

The org. phase was collected and the solvent was removed in vacuum. The oil was taken up in heptane and the solution was filtered once. The heptane was removed in vacuum.

The residual oil was treated for 30 min with 30 mL dichlormethane (DCM) and 50 mL trifluoroacetic acid (TFA), after which the solvent was again removed in vacuum. The oil was taken up in DCM and washed with 1N HCl. The org. phase was dried over sodiumsulfate.

After removal of the solvent, the oil was purified on silica using ethylacetate with 1% acetic acid.

¹H-NMR (400.13 MHz, CDCl₃) 0.88 (t, 3H), 1.24-1.40 (m, 26H), 1.69 (tt, 2H), 2.59 (m, 2H), 2.79 (m, 2H), 3.62-3.69 (m, 8H), 3.72-3.79 (m, 4H), 4.10 (dt, 2H), 4.18-4.36 (m, 4H).

### Example 14

### Synthesis of (3-(2-{2-[2-(Hexadecyloxy-hydroxy-phosphoryloxy)-ethoxy]-ethoxy}-ethoxy)-propionyl)-Ser-Nle-c[Glu-Hyp-D-Phe-Arg-Trp-Lys]-NH2 (B)

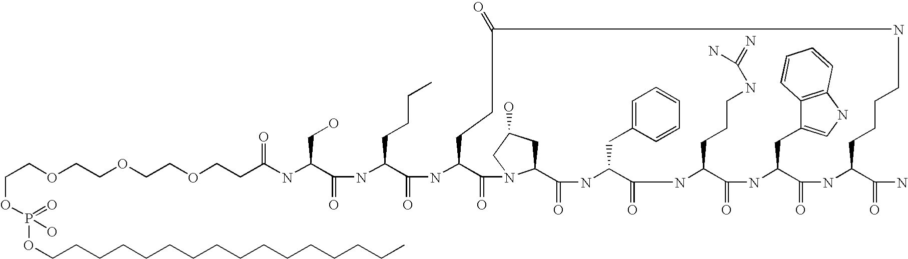

The peptide sequence was synthesised and cyclised as described in WO 2004/099246. After the removal of the fmoc-protecting group at the N-terminal under standard conditions (2 g resin, loading 0.47 mmol/g), the new phosphor-protractor was coupled by dissolving 3 equivalents (A) prepared as described in example 13, 3 equi. HOBt and 3 DIC in 20 mL NMP and adding the mixture to the resin and shaking over night.

The cyano ethyl protecting group was removed by applying standard defmoc condition (2×20% piperidine in NMP). The peptide was then cleaved from the resin using standard cleaving conditions and purified on reverse-phase HPLC (acetonitril/water 0.1% TFA).

### Example 15

### Synthesis of (3-(2-{2-[2-(Hexadecyloxy-hydroxy-phosphoryloxy)-ethoxy]-ethoxy}-ethoxy)-propionyl)-Arg-Nle-c[Glu-Hyp-D-Phe-Arg-Trp-Lys]-NH2 (C)

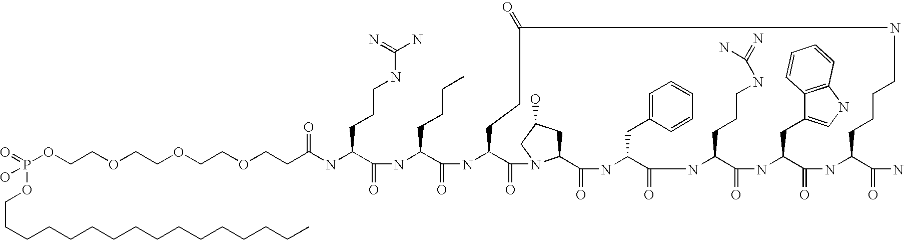

This peptide was synthesised as described for (B) prepared in example 14 using 1.7 equi. of (A) prepared as described in example 13, 1.7 equi. HOBt, 1.7 equi. of DIC using 2 g resin, loading 0.47 mmol/g.

LC-MS: m/z: 1637.3 (M+1), 819.5 ((M+2)/2), 546.7 ((M+3)/3).

### Example 16

### Synthesis of N-Epsilon26-(3-(2-{2-[2-(Hexadecyloxy-hydroxy-phosphoryloxy)-ethoxy]-ethoxy}-ethoxy)-propionyl)-[Aib8, Arg34]GLP-1(7-37) (D)

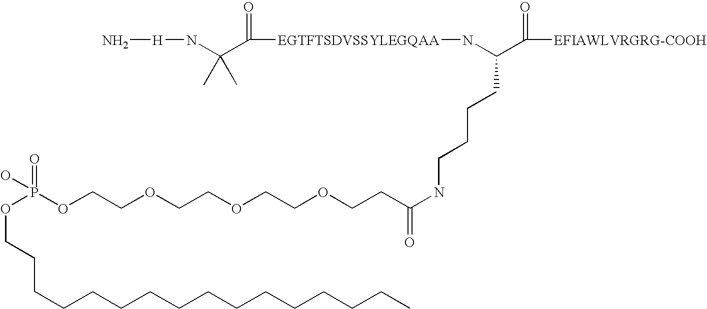

[Aib8, Arg34]GLP-1(7-37) was prepared by standard Fmoc solid phase synthesis, using the following three, backbone-protected dipeptides: Fmoc-Arg(pbf)-(tmob)-Gly-OH, Fmoc-Glu(OtBu)-(tmob)-Gly-OH, and Fmoc-Val-Ser(ψMe,Mepro)-OH and Fmoc-Lys(Mtt)-OH in position 26. After removal of the methyltriphenylmethyl (MTT) from the Lys(26) under standard conditions (Novabiochem catalogue) the phosphor protractor (A) was coupled and deprotected as described above (example 14) using 4 equi. (A), 4 equi. HOBt, 4 equi. DIC in 20 mL NMP using 0.4 g resin, loading 0.62 mmol/g.

LC-MS: m/z: 1954.3 ((M+2)/2), 1303.6 ((M+3)/3), 978.3 ((M+4)/4).

1. A compound of the general formula I: 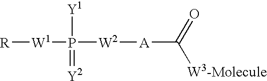
wherein
A represents —(CH₂)₁₋₂₀— in which one or more methylene groups are optionally replaced by a diradical selected from the group consisting of —O—, —S—, —NH—, and —CH═CH— and which is optionally substituted with one or more substituent(s) selected from the group consisting of C₁₋₆-alkyl, C₁₋₆-alkoxy and carboxy,
R represents C₁₋₂₀-alkyl- in which one or more methylene groups are optionally replaced by a diradical selected from the group consisting of —O—, —S—, —NH—, and —CH═CH— and which is optionally substituted with one or more substituent(s) (i) selected from the group consisting of aryl, haloaryl, cyanoaryl, heteroaryl, C₃₋₁₀-cycloalkyl, aryl-C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl; or (ii) C₃₋₁₀-cycloalkyl- optionally substituted with one or more substituent(s) selected from the group consisting of aryl, haloaryl, cyanoaryl, heteroaryl, C₃₋₁₀-cycloalkyl, carboxyl, 5-tetrazolyl, acylaminosulfonyl, sulfonylaminocarbonyl, and a straight or branched C₁₋₆-alkyl,
W¹ and W² independently represent —O—, —CH₂—, or —S—,
Y¹ represents —OH or —SH,
Y² represents ═O or ═S,
W³ represents a bond or a spacer, and
the term ‘molecule’ represents a fragment obtained by formal abstraction of a hydrogen atom from an amino group, a hydroxy group, or a mercapto group of a therapeutically effective polypeptide,
with the proviso that at least either
(I) A represents —(CH₂)₁₁₋₂₀— in which one or more methylene groups are optionally replaced by a diradical selected from the group consisting of —O—, —S—, —NH—, and —CH═CH— and which is optionally substituted with one or more substituent(s) selected from the group consisting of C₁₋₆-alkyl, C₁₋₆-alkoxy and carboxy, or
(II) R represents C₁₁₋₂₀-alkyl- in which one or more methylene groups are optionally replaced by a diradical selected from the group consisting of —O—, —S—, —NH—, and —CH═CH— and which is optionally substituted with one or more substituent(s) selected from the group consisting of aryl, haloaryl, cyanoaryl, heteroaryl, C₃₋₁₀-cycloalkyl, aryl-C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl.

2. A compound of the general formula II 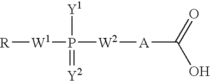
wherein
A represents —(CH₂)₁₋₂₀— in which one or more methylene groups are optionally replaced by a diradical selected from the group consisting of —O—, —S—, —NH—, and —CH═CH— and which is optionally substituted with one or more substituent(s) selected from the group consisting of C₁₋₆-alkyl, C₁₋₆-alkoxy, or carboxy,
R represents C₁₋₂₀-alkyl- in which one or more methylene groups are optionally replaced by (A) a diradical selected from the group consisting of —O—, —S—, —NH—, and —CH═CH— and which is optionally substituted with one or more substituent(s) selected from the group consisting of aryl, haloaryl, cyanoaryl, heteroaryl, C₃₋₁₀-cycloalkyl, aryl-C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl; or (B) C₃₋₁₀-cycloalkyl- optionally substituted with one or more substituent(s) selected from the group consisting of aryl, haloaryl, cyanoaryl, heteroaryl, C₃₋₁₀-cycloalkyl, carboxyl, 5-tetrazolyl, acylaminosulfonyl, sulfonylaminocarbonyl, and a straight or branched C₁₋₆-alkyl,
W¹ and W² independently represent —O—, —CH₂— or —S—,
Y¹ represents —OH or —SH, and
Y² represents ═O or ═S,
with the proviso that
(A) at least either A represents —(CH₂)₁₁₋₂₀— in which one or more methylene groups are optionally replaced by a diradical selected from the group consisting of —O—, —S—, —NH—, and —CH═CH— and which is optionally substituted with one or more substituent(s) selected from the group consisting of C₁₋₆-alkyl, C₁₋₆-alkoxy and carboxy, or
(B) R represents C₁₁₋₂₀-alkyl- in which one or more methylene groups are optionally replaced by a diradical selected from the group consisting of —O—, —S—, —NH—, and —CH═CH— and which is optionally substituted with one or more substituent(s) selected from the group consisting of aryl, haloaryl, cyanoaryl, heteroaryl, C₃₋₁₀-cycloalkyl, aryl C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl.

3. The compound according to claim 1, wherein A represents —(CH₂)₁₁₋₂₀— in which one or more methylene groups are optionally replaced by a diradical selected from the group consisting of —O—, —S—, —NH—, and —CH═CH— and which is optionally substituted with one or more substituent(s) selected from the group consisting of C₁₋₆-alkyl, C₁₋₆-alkoxy, or carboxy.

4. The compound according to claim 1, wherein R represents C₁₋₂₀-alkyl- in which one or more methylene groups are optionally replaced by a diradical selected from the group consisting of —O—, —S—, —NH—, and —CH═CH— and which is optionally substituted with one or more substituent(s) selected from the group consisting of aryl, haloaryl, cyanoaryl, heteroaryl, C₃₋₁₀-cycloalkyl, aryl-C₃₋₁₀-cycloalkyl, diaryl-C₃₋₁₀-cycloalkyl, carboxyl, 5-tetrazolyl, acylaminosulfonyl, sulfonylaminocarbonyl, and a straight or branched C₁₋₆-alkyl.

5. The compound according to claim 1, wherein A represents —(CH₂)₁₁₋₂₀— in which one or more methylene groups are optionally replaced by a diradical selected from the group consisting of —O—, —S—, —NH—, and —CH═CH—.

6. The compound according to claim 1, wherein A represents —(CH₂)₁₁₋₂₀—.

7. The compound according to claim 1, wherein A represents —(CH₂)₁₁₋₁₈—.

8. The compound according to claim 1, wherein R represents C₁₋₂₀-alkyl- in which one or more methylene groups are optionally replaced by a diradical selected from the group consisting of —O—, —S—, —NH— and —CH═CH—.

9. The compound according to claim 1, wherein R represents C₁₋₂₀-alkyl-.

10. The compound according to claim 1, wherein R represents C₁₁₋₂₀-alkyl-.

11. The compound according to claim 1, wherein R represents C₁₄₋₁₈-alkyl-.

12. The compound according to claim 1, wherein A represents —(CH₂)₁₋₂₀— in which one or more methylene groups are replaced by —O—.

13. The compound according claim 1, wherein A represents —(CH₂)₁₋₂₀— in which one or more methylene groups are replaced by —S—.

14. The compound according to claim 1, wherein A represents —(CH₂)₁₋₂₀— in which one or more methylene groups are replaced by —CH═CH—.

15-17. (canceled)

18. The compound according to claim 1, wherein W¹ represents —O—.

19. The compound according to claim 1, wherein W² represents —O— or —CH₂—.

20. The compound according to claim 1, wherein Y¹ represents —OH.

21. The compound according to claim 1, wherein Y² represents ═O.

22. The compound according to claim 1, wherein the spacer W³ is selected from the group consisting of oligo(ethylene glycol), an amino acid or a combination thereof.

23. The compound according to claim 1, wherein the spacer W³ is selected from the group consisting of:

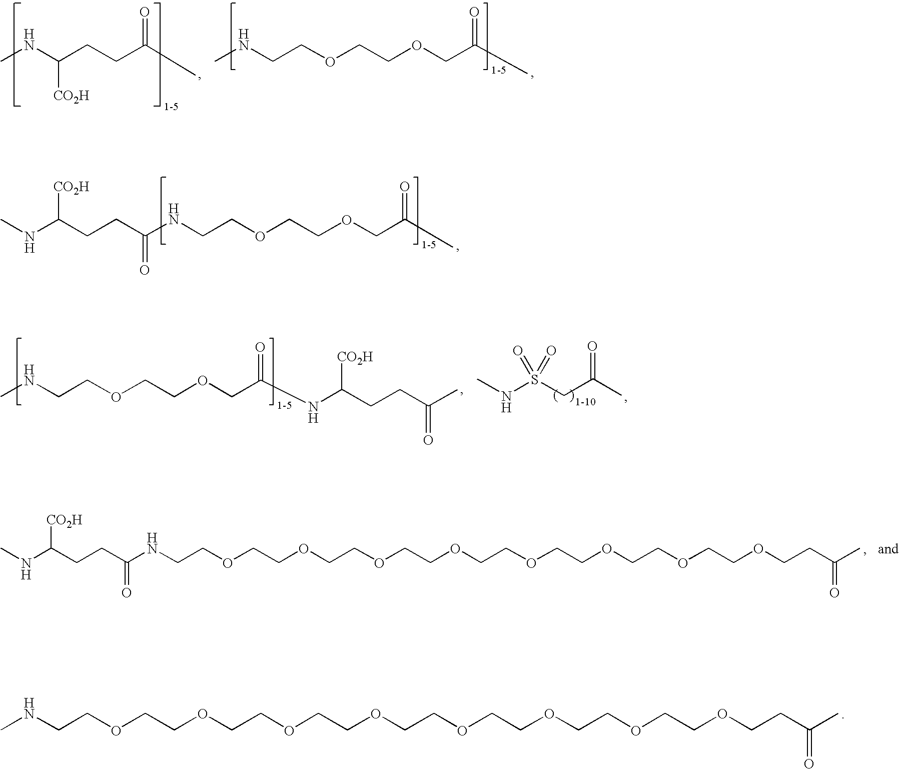

24. The compound according to claim 1, wherein said polypeptide comprises the amino acid sequence of the formula (III):
Xaa₇-Xaa₈-Glu-Gly-Thr-Phe-Thr-Ser-Asp-Xaa₁₆-Ser-Xaa₁₈-Xaa₁₉-Xaa₂₀-Glu-Xaa₂₂-Xaa₂₃-Ala-Xaa₂₅-Xaa₂₆-Xaa₂₇-Phe-Ile-Xaa₃₀-Trp-Leu-Xaa₃₃-Xaa₃₄-Xaa₃₅-Xaa₃₆-Xaa₃₇-Xaa₃₈-Xaa₃₉-Xaa₄₀-Xaa₄₁-Xaa₄₂-Xaa₄₃-Xaa₄₄-Xaa₄₅-Xaa₄₆ Formula (III) (SEQ ID No: 3)
wherein
Xaa₇ is L-histidine, D-histidine, desamino-histidine, 2-amino-3-(2-aminoimidazol-4-yl)propionic acid, β-hydroxy-histidine, homohistidine, N^{α}-acetyl-histidine, α-fluoromethyl-histidine, α-methyl-histidine, 3-pyridylalanine, 2-pyridylalanine, or 4-pyridylalanine;
Xaa₈ is Ala, Gly, Val, Leu, Ile, Lys, Aib, 1-aminocyclopropanecarboxylic acid, 1-aminocyclobutanecarboxylic acid, 1-aminocyclopentanecarboxylic acid, 1-aminocyclohexanecarboxylic acid, 1-aminocycloheptanecarboxylic acid, or 1-aminocyclooctanecarboxylic acid;
Xaa₁₆ is Val or Leu;
Xaa₁₈ is Ser, Lys, or Arg;
Xaa₁₉ is Tyr or Gln;
Xaa₂₀ is Leu or Met;
Xaa₂₂ is Gly, Glu, or Aib;
Xaa₂₃ is Gln, Glu, Lys, or Arg;
Xaa₂₅ is Ala or Val;
Xaa₂₆ is Lys, Glu, or Arg;
Xaa₂₇ is Glu or Leu;
Xaa₃₀ is Ala, Glu, or Arg;
Xaa₃₃ is Val or Lys;
Xaa₃₄ is Lys, Glu, Asn, or Arg;
Xaa₃₅ is Gly, or Aib;
Xaa₃₆ is Arg, Gly, or Lys;
Xaa₃₇ is Gly, Ala, Glu, Pro, Lys, amide, or is absent;
Xaa₃₈ is Lys, Ser, amide, or is absent;
Xaa₃₉ is Ser, Lys, amide, or is absent;
Xaa₄₀ is Gly, amide, or is absent;
Xaa₄₁ is Ala, amide, or is absent;
Xaa₄₂ is Pro, amide, or is absent;
Xaa₄₃ is Pro, amide, or is absent;
Xaa₄₄ is Pro, amide, or is absent;
Xaa₄₅ is Ser, amide, or is absent;
Xaa₄₆ is amide or is absent;
provided that if Xaa₃₈, Xaa₃₉, Xaa₄₀, Xaa₄₁, Xaa₄₂, Xaa₄₃, Xaa₄₄, Xaa₄₅ or Xaa₄₆ is absent then each amino acid residue downstream is also absent.

25. The compound according to claim 1, wherein said polypeptide is selected from GLP-1(7-35), GLP-1(7-36), GLP-1(7-36)-amide, GLP-1(7-37), GLP-1(7-38), GLP-1(7-39), GLP-1(7-40), GLP-1(7-41) or an analogue thereof.

26. The compound according to claim 25 wherein said polypeptide is selected from the group consisting of Arg³⁴GLP-1(7-37), Lys³⁸Arg^{26,34}GLP-1(7-38), Lys³⁸Arg^{26,34}GLP-1(7-38)-OH, Lys³⁶Arg^{26,34}GLP-1(7-36), Aib^{8,22,35}GLP-1(7-37), Aib^{8,35}GLP-1(7-37), Aib^{8,22}GLP-1(7-37), Aib^{8,22,35}Arg^{26,34}Lys³⁸GLP-1(7-38), Aib^{8,35}Arg^{26,34}Lys³⁸GLP-1(7-38), Aib^{8,22}Arg^{26,34}Lys³⁸GLP-1(7-38), Aib^{8,22,35}Arg^{26,34}Lys³⁸GLP-1(7-38), Aib^{8,35}Arg^{26,34}Lys³⁸GLP-1(7-38), Aib^{8,22,35}Arg²⁶Lys³⁸GLP-1(7-38), Aib^{8,35}Arg²⁶Lys³⁸GLP-1(7-38), Aib^{8,22}Arg²⁶Lys³⁸GLP-1(7-38), Aib^{8,22,35}Arg34Lys³⁸GLP-1(7-38), Aib8,35Arg34 Lys³⁸GLP-1(7-38), Aib^{8,22}Arg³⁴Lys³⁸GLP-1(7-38), Aib^{8,22,35}Ala³⁷Lys³⁸GLP-1(7-38), Aib^{8,35}Ala³⁷Lys³⁸GLP-1(7-38), Aib^{8,22}Ala³⁷Lys³⁸GLP-1(7-38), and Aib^{8,22,35} Lys³⁷GLP-1(7-37), Aib^{8,35}Lys³⁷GLP-1(7-37) and Aib^{8,22}Lys³⁷GLP-1(7-38).

27. The compound according to claim 1, wherein said polypeptide is ZP-10, i.e. HGEGTFTSDLSKQMEEEAVRLFIEWLKNGGPSSGAPPSKKKKKK-amide (SEQ ID No. 5).

28. The compound according to claim 1, wherein the polypeptide is a polypeptide with formula V
R¹-X-X¹-X²-X³-X⁴-X⁵-X⁶-X⁷-X⁸-X⁹-X¹⁰-X¹¹-R² [V]
wherein
R¹, which is bonded to an N-terminal amino group is either absent or represents C₁₋₄alkanoyl;
X represents a bond or an amino acid, a di- or tri-peptide residue, wherein the amino acid(s) may be natural or synthetic;
X¹ represents a bond or an amino acid residue with a functional group in the side chain to which a protracting group may be attached;
X² represents a bond or an amino acid, di-, tri- or tetra-peptide residue, wherein the amino acid(s) may be natural or synthetic;
X³ represents a bond or an amino acid residue optionally capable of making a bridge to X¹⁰;
X⁴ represents a bond or an amino acid or di-peptide residue, wherein the amino acid(s) may be natural or synthetic;
X⁵ represents an amino acid residue selected from His, Ala, Nle, Met, Met(O), Met(O₂), Gln, Gln(ε-alkyl), Gln(ε-aryl), Asn, Asn(ε-alkyl), Asn(ε-aryl), Ser, Thr, Cys, F-Pro, Pro, Hyp, (S)-1,2,3,4-tetrahydroisoquinoline-3-carboxylic acid, Trp, 1-naphthylalanine, 2-naphthylalanine, 2-pyridylalanine, 3-pyridylalanine, 4-pyridylalanine, 2-thienylalanine, 3-thienylalanine, 4-thiazolylalanine, 2-furylalanine, 3-furylalanine, or Phe, wherein the phenyl moiety of said Phe is optionally substituted by halogen, hydroxyl, alkoxy, nitro, benzoyl, methyl, trifluoromethyl or cyano;
X⁶ represents (D)-Phe, wherein the phenyl moiety of said (D)-Phe is optionally substituted with halogen, hydroxy, alkoxy, nitro, methyl, trifluoromethyl or cyano;
X⁷ represents Arg;
X⁸ represents Trp or 2-naphthylalanine;
X⁹ represents a bond or an amino acid, or di-peptide residue, wherein the amino acid(s) may be natural or synthetic;
X¹⁰ represents a bond or an amino acid residue optionally capable of making a bridge to X³;
X¹¹ represents a bond, an amino acid or a di-peptide, wherein the amino acid(s) may be natural or synthetic;
R² represents —OH or —NRR′, wherein R and R′ independently represent hydrogen, C₁₋₈alkyl, C₂₋₈alkenyl or C₂₋₈alkynyl;
wherein the peptide of formula I is optionally cyclized from X³ to X¹⁰ via a lactame or a disulfide bridge;
with the proviso that the polypeptide of formula V comprises at least 7 amino acid residues;
and any pharmaceutically acceptable salt, solvate or hydrate thereof.

29. A compound selected from the group consisting of
N-ε²⁶-((S)-4-[16-{(hydroxy)(octadecyloxy)phosphoryloxy}hexadecanoylamino]-4-carboxybutyryl)[Aib8, Arg34]GLP-1(7-37),
N-ε²⁶-((S)-4-[16-{(hydroxy)(pentyloxy)phosphoryloxy}hexadecanoylamino]-4-carboxybutyryl)[Aib8, Arg34]GLP-1(7-37),
N-ε²⁶-((S)-4-[16-{(hydroxy)(dodecyloxy)phosphoryloxy}hexadecanoylamino]-4-carboxybutyryl)[Aib8, Arg34]GLP-1(7-37),
N-ε²⁶-((S)-4-[16-{(hydroxy)(methoxy)phosphoryl}nonadecanoylamino]-4-carboxybutyryl)[Aib8, Arg34]GLP-1(7-37),
(3-(2-{2-[2-(Hexadecyloxy-hydroxy-phosphoryloxy)-ethoxy]-ethoxy}-ethoxy)-propionyl)-Ser-Nle-c[Glu-Hyp-D-Phe-Arg-Trp-Lys]-NH₂,
(3-(2-{2-[2-(Hexadecyloxy-hydroxy-phosphoryloxy)-ethoxy]-ethoxy}-ethoxy)-propionyl)-Arg-Nle-c[Glu-Hyp-D-Phe-Arg-Trp-Lys]-NH₂, and
N-Epsilon26-(3-(2-{2-[2-(Hexadecyloxy-hydroxy-phosphoryloxy)-ethoxy]-ethoxy}-ethoxy)-propionyl)-[Aib8, Arg34]GLP-1(7-37).
